(19) United States
(12) Patent Application Publication
(10) Pub. No.: US 2011/0158907 A1
(43) Pub. Date: Jun. 30, 2011

(54) DIPHENYL-HETEROARYL DERIVATIVES AND THEIR USE FOR BINDING AND IMAGING AMYLOID PLAQUES

(75) Inventors: Hank F. Kung, Wynnewood, PA (US); Mei-Ping Kung, Wynnewood, PA (US); Wenchao Qu, Philadelphia, PA (US)

(73) Assignee: The Trustees of the Univeristy of Pennsylvania, Philadelphia, PA (US)

(21) Appl. No.: 12/595,131

(22) PCT Filed: Apr. 18, 2008
(86) PCT No.: PCT/US08/60710

§ 371 Date: Mar. 25, 2010

Related U.S. Application Data
(60) Provisional application No. 60/907,841, filed on Apr. 19, 2007.
(60) Provisional application No. 60/929,806, filed on Jul. 13, 2007.

(51) Int. Cl. A61K 51/00; C07D 249/06; C07F 13/00; A61K 31/4192; A61P 25/28

(52) U.S. Cl. 424/1.85; 548/255; 534/14; 514/359

(57) ABSTRACT

This invention relates to a method of imaging amyloid deposits and to diphenyl-heteroaryl compounds, and methods of making radiolabeled diphenyl-heteroaryl compounds useful in imaging amyloid deposits. This invention also relates to compounds, and methods of making compounds for inhibiting the aggregation of amyloid proteins to form amyloid deposits, and a method of delivering a therapeutic agent to amyloid deposits.

### CROSS REFERENCE TO RELATED APPLICATIONS

This application claims the benefit of U.S. Provisional Application No. 60/907,841, filed Apr. 19, 2007, the entirety of which is incorporated herein by reference.

### STATEMENT REGARDING FEDERALLY-SPONSORED RESEARCH

Part of the work performed during development of this invention utilized U.S. Government funds. The U.S. Government has certain rights in this invention under grant numbers AG-022559 and AG-021868 awarded by the National Institutes of Health.

### FIELD OF THE INVENTION

This invention relates to diphenyl-heteroaryl compounds, the uses thereof in diagnostic imaging and inhibiting amyloid-β aggregation, and methods of making these compounds.

### BACKGROUND OF THE INVENTION

Alzheimer's disease (AD) is a progressive neurodegenerative disorder characterized by cognitive decline, irreversible memory loss, disorientation, and language impairment. Postmortem examination of AD brain sections reveals abundant senile plaques (SPs) composed of amyloid-β (Aβ) peptides and numerous neurofibrillary tangles (NFTs) formed by filaments of highly phosphorylated tau proteins (for recent reviews and additional citations see Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, NY (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372).

Amyloidosis is a condition characterized by the accumulation of various insoluble, fibrillar proteins in the tissues of a patient. An amyloid deposit is formed by the aggregation of amyloid proteins, followed by the further combination of aggregates and/or amyloid proteins. Formation and accumulation of aggregates of β amyloid (Aβ peptides in the brain are critical factors in the development and progression of AD.

In addition to the role of amyloid deposits in Alzheimer's disease, the presence of amyloid deposits has been shown in diseases such as Mediterranean fever, Muckle-Wells syndrome, idiopathic myeloma, amyloid polyneuropathy, amyloid cardiomyopathy, systemic senile amyloidosis, amyloid polyneuropathy, hereditary cerebral hemorrhage with amyloidosis, Down's syndrome, Scrapie, Creutzfeldt-Jacob disease, Kuru, Gerstamnn-Straussler-Scheinker syndrome, medullary carcinoma of the thyroid, Isolated atrial amyloid, β₂-microglobulin amyloid in dialysis patients, inclusion body myositis, β₂-amyloid deposits in muscle wasting disease, and Islets of Langerhans diabetes Type II insulinoma.

The fibrillar aggregates of amyloid peptides, Aβ₁₋₄₀ and Aβ₁₋₄₂, are major metabolic peptides derived from amyloid precursor protein found in senile plaques and cerebrovascular amyloid deposits in AD patients (Xia, W., et al., J. Proc. Natl. Acad. Sci. U.S.A. 97:9299-9304 (2000)). Prevention and reversal of Aβ plaque formation are being targeted as a treatment for this disease (Selkoe, D., J. JAMA 283:1615-1617 (2000); Wolfe, M. S., et al., J. Med. Chem. 41:6-9 (1998); Skovronsky, D. M., and Lee, V. M., Trends Pharmacol. Sci. 21:161-163 (2000)).

Familial AD (FAD) is caused by multiple mutations in the A precursor protein (APP), presenilin 1 (PS1) and presenilin 2 (PS2) genes (Ginsberg, S. D., et al., “Molecular Pathology of Alzheimer's Disease and Related Disorders,” in Cerebral Cortex: Neurodegenerative and Age-related Changes in Structure and Function of Cerebral Cortex, Kluwer Academic/Plenum, NY (1999), pp. 603-654; Vogelsberg-Ragaglia, V., et al., “Cell Biology of Tau and Cytoskeletal Pathology in Alzheimer's Disease,” Alzheimer's Disease, Lippincot, Williams & Wilkins, Philadelphia, Pa. (1999), pp. 359-372).

While the exact mechanisms underlying AD are not fully understood, all pathogenic FAD mutations studied thus far increase production of the more amyloidogenic 42-43 amino-acid long form of the Aβ peptide. Thus, at least in FAD, dysregulation of Aβ production appears to be sufficient to induce a cascade of events leading to neurodegeneration. Indeed, the amyloid cascade hypothesis suggests that formation of extracellular fibrillar Aβ aggregates in the brain may be a pivotal event in AD pathogenesis (Selkoe, D. J., “Biology of β-amyloid Precursor Protein and the Mechanism of Alzheimer's Disease,” Alzheimer's Disease, Lippincot Williams & Wilkins, Philadelphia, Pa. (1999), pp. 293-310; Selkoe, D. J., J. Am. Med. Assoc. 283:1615-1617 (2000); Naslund, J., et al., J. Am. Med. Assoc. 283:1571-1577 (2000); Golde, T. E., et al., Biochimica et Biophysica Acta 1502:172-187 (2000)).

Various approaches in trying to inhibit the production and reduce the accumulation of fibrillar Aβ in the brain are currently being evaluated as potential therapies for AD (Skovronsky, D. M. and Lee, V. M., Trends Pharmacol. Sci. 21:161-163 (2000); Vassar, R., et al., Science 286:735-741 (1999); Wolfe, M. S., et al., J. Med. Chem. 41:6-9 (1998); Moore, C. L., et al., J. Med. Chem. 43:3434-3442 (2000); Findeis, M. A., Biochimica et Biophysica Acta 1502:76-84 (2000); Kuner, P., Bohrmann, et al., J. Biol. Chem. 275:1673-1678 (2000)). It is therefore of interest to develop ligands that specifically bind fibrillar Aβ aggregates. Since extracellular SPs are accessible targets, these new ligands could be used as in vivo diagnostic tools and as probes to visualize the progressive deposition of Aβ in studies of AD amyloidogenesis in living patients.

To this end, several interesting approaches for developing fibrillar Aβ aggregate-specific ligands have been reported (Ashburn, T. T., et al., Chem. Biol. 3:351-358 (1996); Han, G., et al., J. Am. Chem. Soc. 118:4506-4507 (1996); Klunk, W. E., et al., Biol. Psychiatry 35:627 (1994); Klunk, W. E., et al., Neurobiol. Aging 16:541-548 (1995); Klunk, W. E., et al., Society for Neuroscience Abstract 23:1638 (1997); Mathis, C. A., et al., Proc. XIIth Intl. Symp. Radiopharm. Chem., Uppsala, Sweden:94-95 (1997); Lorenzo, A. and Yankner, B. A., Proc. Natl. Acad. Sci. U.S.A. 91:12243-12247 (1994); Zhen, W., et al., J. Med. Chem. 42:2805-2815 (1999)). The most attractive approach is based on highly conjugated chrysamine-G (CG) and Congo red (CR), and the latter has been used for fluorescent staining of SPs and NFTs in postmortem AD brain sections (Ashburn, T. T., et al., Chem. Biol. 3:351-358 (1996); Klunk, W. E., et al., J. Histochem. Cytochem. 37:1273-1281 (1989)). Thg inhibition constants (Kᵢ) for binding to fibrillar Aβ aggregates of CR, CG, and 3′-bromo- and 3′-iodo derivatives of CG are 2,800, 370, 300 and 250 nM, respectively (Mathis, C. A., et al., Proc. XIIth Intl. Symp. Radiopharm. Chem., Uppsala, Sweden:94-95 (1997)). These compounds have been shown to bind selectively to Aβ (1-40) peptide aggregates in vitro as well as to fibrillar Aβ deposits in AD brain sections (Mathis, C. A., et al., Proc. XIIth Intl. Symp. Radiopharm. Chem., Uppsala, Sweden:94-95 (1997)).

There are several potential benefits of imaging Aβ aggregates in the brain. The imaging technique will improve diagnosis by identifying potential patients with excess Aβ plaques in the brain; therefore, they may be likely to develop Alzheimer's disease. It will also be useful to monitor the progression of the disease. When anti-plaque drug treatments become available, imaging Aβ plaques in the brain may provide an essential tool for monitoring treatment. Thus, a simple, noninvasive method for detecting and quantitating amyloid deposits in a patient has been eagerly sought. Presently, detection of amyloid deposits involves histological analysis of biopsy or autopsy materials. Both methods have drawbacks. For example, an autopsy can only be used for a postmortem diagnosis.

The direct imaging of amyloid deposits in vivo is difficult, as the deposits have many of the same physical properties (e.g., density and water content) as normal tissues. Attempts to image amyloid deposits using magnetic resonance imaging (MRI) and computer-assisted tomography (CAT) have been disappointing and have detected amyloid deposits only under certain favorable conditions. In addition, efforts to label amyloid deposits with antibodies, serum amyloid P protein, or other probe molecules have provided some selectivity on the periphery of tissues, but have provided for poor imaging of tissue interiors.

Potential ligands for detecting Aβ aggregates in the living brain must cross the intact blood-brain barrier. Thus brain uptake can be improved by using ligands with relatively smaller molecular size (compared to Congo Red) and increased lipophilicity. Highly conjugated thioflavins (S and T) are commonly used as dyes for staining the Aβ aggregates in the AD brain (Elhaddaoui, A., et al., Biospectroscopy 1:351-356 (1995)).

A highly lipophilic tracer, [¹⁸F]FDDNP, for binding both tangles (mainly composed of hyperphosphorylated tau protein) and plaques (containing Aβ protein aggregates) has been reported. (Shoghi-Jadid K, et al., Am J Geriatr Psychiatry. 2002; 10:24-35). Using positron-emission tomography (PET), it was reported that this tracer specifically labeled deposits of plaques and tangles in nine AD patients and seven comparison subjects. (Nordberg A. Lancet Neurol. 2004; 3:519-27). Using a novel pharmacokinetic analysis procedure called the relative residence time of the brain region of interest versus the pons, differences between AD patients and comparison subjects were demonstrated. The relative residence time was significantly higher in AD patients. This is further complicated by an intriguing finding that FDDNP competes with some NSAIDs for binding to Aβ fibrils in vitro and to Aβ plaques ex vivo (Agdeppa E D, et al. 2001; Agdeppa E D, et al., Neuroscience. 2003; 117:723-30).

Imaging β-amyloid in the brain of AD patients by using a benzothiazole aniline derivative, [¹¹C6-OH-BTA-1 (also referred to as [¹¹C]PIB), was recently reported. (Mathis C A, et al., Curr Pharm Des. 2004; 10:1469-92; Mathis C A, et al., Arch. Neurol. 2005, 62:196-200.). Contrary to that observed for [¹⁸FDDNP, [¹¹C]6-OH-BTA-1 binds specifically to fibrillar Aβ in vivo. Patients with diagnosed mild AD showed marked retention of [¹¹C]6-OH-BTA-1 in the cortex, known to contain large amounts of amyloid deposits in AD. In the AD patient group, [¹¹C]6-OH-BTA-1 retention was increased most prominently in the frontal cortex. Large increases also were observed in parietal, temporal, and occipital cortices and in the striatum. [¹¹C]6-OH-BTA-1 retention was equivalent in AD patients and comparison subjects in areas known to be relatively unaffected by amyloid deposition (such as subcortical white matter, pons, and cerebellum). Recently, another ¹¹C labeled Aβ plaque-targeting probe, a stilbene derivative-[¹¹C]SB-13, has been studied. In vitro binding using the [³H]SB-13 suggests that the compound showed excellent binding affinity and binding can be clearly measured in the cortical gray matter, but not in the white matter of AD cases. (Kung M-P, et al., Brain Res. 2004; 1025:89-105. There was a very low specific binding in cortical tissue homogenates of control brains. The Kd values of [³H]SB-13 in AD cortical homogenates were 2.4±0.2 nM. High binding capacity and comparable values were observed (14-45 pmol/mg protein) (Id.). As expected, in AD patients [¹¹C]SB-13 displayed a high accumulation in the frontal cortex (presumably an area containing a high density of Aβ plaques) in mild to moderate AD patients, but not in age-matched control subjects. (Verhoeff N P, et al., Am J Geriatr Psychiatry. 2004; 12:584-95).

It would be useful to have a noninvasive technique for imaging and quantitating amyloid deposits in a patient. In addition, it would be useful to have compounds that inhibit the aggregation of amyloid proteins to form amyloid deposits and a method for determining a compound's ability to inhibit amyloid protein aggregation.

### SUMMARY OF THE INVENTION

The present invention provides novel compounds of Formulae I, I′, II, I″ and I′″. The present invention also provides diagnostic compositions comprising radiolabeled compounds of Formulae. I, I′, II, I″ and I′″ and a pharmaceutically acceptable carrier or diluent.

The invention further provides methods of imaging amyloid deposits, the methods comprising introducing into a patient a detectable quantity of a labeled compound of Formulae I, I′, II, I″ or I′″ or a pharmaceutically acceptable salt, ester, amide or prodrug thereof.

The present invention also provides methods for inhibiting the aggregation of amyloid proteins, the methods comprising administering to a mammal an amyloid inhibiting amount of a compound of Formulae I, I′, II, I″ or I′″ or a pharmaceutically acceptable salt, ester, amide, or prodrug thereof.

A further aspect of this invention is directed to methods and intermediates useful for synthesizing the amyloid inhibiting and imaging compounds of Formulae I, I′, II, I″ and I′″ described herein.

### BRIEF DESCRIPTION OF THE DRAWINGS

FIG. 1 depicts preferred triazole embodiments of the present invention.

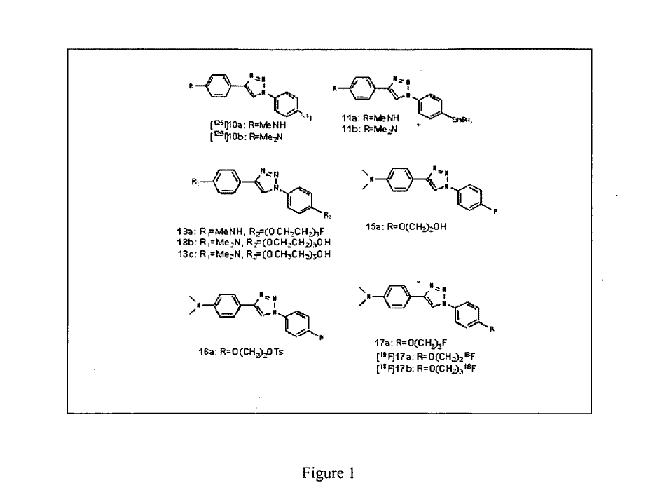

FIG. 2 depicts preferred triazole embodiments of the present invention and certain of their respective binding data.

FIG. 3 depicts HPLC profiles of preferred embodiments of the present invention. HPLC condition: Agilent 1100 series; Gemini C-18 analytical column (4.6×250 mm, 5 mm), CH₃CN/ammonium formate (10 mM) 8/2,1 mL/min 265 nm tR=(UV) 4.5 min, (Y) 4.8 min. The slight difference in retention time between the radioactive peak and the UV peak is due to the configuration of detector system).

FIG. 4 depicts brain uptakes and washouts of preferred embodiments of preferred radiolabeled triazole probes of the present invention in normal mice.

FIG. 5 depicts in vitro film autoradiography of macroarray brain sections constructed from AD and control cases. The Aβ plaques were clearly visualized with low background labeling with two preferred radiofluorinated probes of the present invention. High white matter labeling was observed with the iodinated probe, in addition to plaque labeling.

FIG. 6 depicts specific binding of preferred embodiments of the present invention to pooled AD and control brain tissue homogenates. Gray and white matters were dissected from the cortical regions. Higher specific binding was detected mainly in gray matter of AD. Relatively low binding was measured in white matter homogenates of AD as well as in gray and white matter homogenates of the control brain.

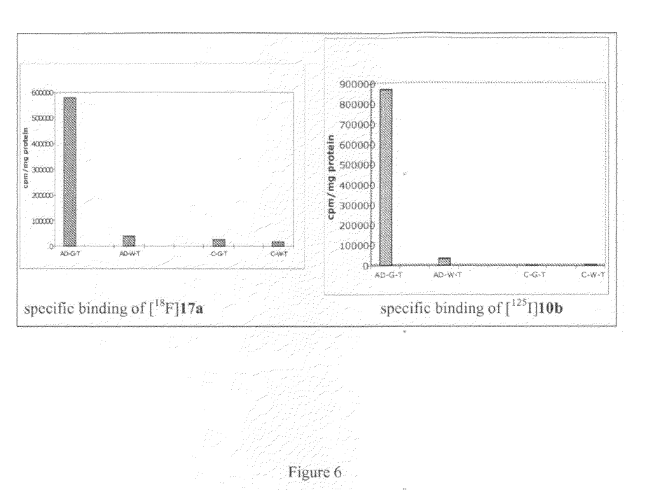

FIG. 7. depicts the Standardized Uptake Value (nCi/cc/mCi injected) of one embodiment of the present invention in cortical matter regions (open circles) and white matter regions (closed circles) of the rhesus monkey brain versus time post injection. Very fast and high uptake is seen in the cortex with rapid washout, along with low non-specific binding and washout in white matter regions

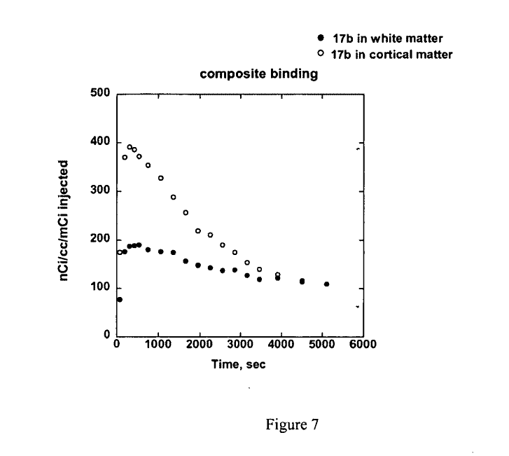

### DETAILED DESCRIPTION OF ILLUSTRATIVE EMBODIMENTS

A compound of Formula I,

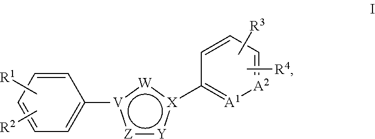

or a pharmaceutically acceptable salt thereof; wherein:
W, Y and Z are each independently CH, N, NH, O or S; provided that at least one of W, Y and Z is N or O;
V and X are independently C or N;
A¹ and A² are independently N, CR³ or CR⁴ as permitted;
R¹ and R² are independently:
   —(CH₂)ₚNR^{a}R^{b}, wherein R^{a} and R^{b} are independently hydrogen, (C₁₋₄)alkyl, hydroxy(C₁₋₄alkyl or halo(C₁₋₄)alkyl, and p is an integer from 0 to 5; hydroxy; (C₁₋₄)alkoxy; hydroxy(C₁₋₄)alkyl; halogen; cyano; hydrogen; nitro; (C₁-C₄)alkyl; halo(C₁-C₄)alkyl; formyl; —NHCO(C₁₋₄ alkyl); —OCO(C₁₋₄ alkyl); or radiohalogen;
R³ is hydrogen or i-vi as shown below:

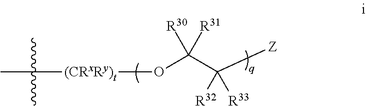

wherein q is an integer from 1 to 10; R^{x} and R^{y} are hydrogen, hydroxy or (C₁₋₄)alkyl; t is 0, 1, 2 or 3; Z is halogen, hydroxy, OTs (tosylate) or amino; and R³⁰, R³¹, R³² and R³³ are in each instance independently hydrogen, hydroxy, (C₁₋₄)alkoxy, (C₁₋₄)alkyl, or hydroxy(C₁₋₄)alkyl;

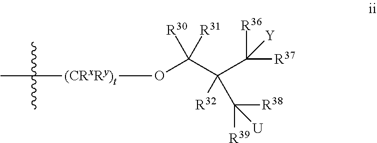

wherein R^{x} and R^{y} are hydrogen, hydroxy or (C₁₋₄)alkyl; t is 0, 1, 2 or 3; Y is halogen, halogen substituted benzoyloxy, halogen substituted phenyl(C₁₋₄)alkyl, halogen substituted aryloxy, or halogen substituted (C₆₋₁₀)aryl; U is hydrogen, hydroxy, halogen, halogen substituted benzoyloxy, halogen substituted phenyl(C₁₋₄)alkyl, halogen substituted aryloxy, or halogen substituted (C₆₋₁₀)aryl; and R³⁴, R³⁵, R³⁶, R³⁷, R³⁸, R³⁹ and R⁴⁰ are in each instance independently hydrogen, halogen, hydroxy, (C₁₋₄)alkoxy, (C₁₋₄)alkyl, or hydroxy(C₁₋₄)alkyl;
R⁴ is hydrogen, halogen, radiohalogen or —(C₁₋₄ alkyl)₃Sn, preferably (Bu)₃Sn;
iii. NR′R″, wherein at least one of R′ and R″ is (CH₂)_{d}X, where X is halogen, preferably F or ¹⁸F, and d is an integer from 1 to 4; the other of R′ and R″ is hydrogen, (C₁₋₄)alkyl, halo(C₁₋₄)alkyl, and hydroxy(C₁₋₄)alkyl;
iv. NR′R″—(C₁₋₄)alkyl, wherein at least one of R′ and R″ is (CH₂)_{d}X, where X is halogen, preferably F or ¹⁸F, and d is an integer from 1 to 4; the other of R′ and R″ is hydrogen, (C₁₋₄)alkyl, halo(C₁₋₄)alkyl, or hydroxy(C₁₋₄)alkyl;
v. halo(C₁₋₄)alkyl; or
vi. an ether (R—O—R) having the following structure: [halo(C₁₋₄)alkyl-O—(C₁₋₄)alkyl]-;

provided that one of R³ and R⁴ is other than hydrogen.

Preferred compounds include those where the halogen, in one or more occurrence on the structure, is a radiolabeled halogen. Also preferred are compounds wherein the halogen is I, ¹²³I, ¹²⁵I, ¹³¹I, Br, ⁷⁶Br, ⁷⁷Br, F or ¹⁸F. Especially preferred compounds are those that contain ¹⁸F. Compounds containing ¹²³I are also especially preferred.

Useful values of R¹ and R² are listed above. Preferably one of R¹ and R² is hydrogen. Other preferred values are hydroxy or NR^{a}R^{b}(CH₂)ₚ—, wherein p is an integer from 0 to 5, and R^{a} and R^{b} are independently hydrogen, C₁₋₄ alkyl or (CH₂)_{d}X, where X is halogen, and d is an integer from 1 to 4 Useful values of p include integers from 0 to 5. Preferably, p is 0, 1 or 2. Most preferably, p is 0 such that R¹ or R² represents NR^{a}R^{b}. In preferred embodiments, R¹ is other than hydrogen and is either in the meta or para position relative to the respective bridge. A preferred value of R¹ is NR^{a}R^{b}, wherein R^{a} and R^{b} are independently hydrogen or (C₁₋₄)alkyl. In this embodiment, it is preferable that the (C₁₋₄)alkyl is methyl. Preferably one of R^{a} and R^{b} is hydrogen, the other is (C₁₋₄)alkyl, such as methyl. Most preferably, both R^{a} and R^{b} are methyl. Another preferred value of R¹ is hydroxy. Also preferred arc any prodrug groups that after administration yield a preferred value of R¹. Such prodrug groups are well-known in the art.

Useful values of R³ include substructures i, ii, iii, iv, v, and vi, as depicted above. In preferred embodiments of Formula I, R³ is either in the meta or para position relative to the respective bridge. Preferably, R³ is substructure i or ii. In these embodiments, useful values of q include integers from one to ten. Preferably, in a compound where R³ is i, q is an integer from 1 to 5. Most preferably, q is 1 to 4, especially 1 to 3. In substructure i, useful values of R³⁰, R³¹, R³² and R³³ independently include hydrogen, hydroxy, C₁₋₄ alkoxy, C₁₋₄ alkyl, and hydroxy(C₁₋₄)alkyl. Preferred compounds include those where one or more of R³⁰, R³¹, R³² and R³³ are hydrogen. More preferred compounds include those where each of R³⁰, R³¹, R³² and R³³ is hydrogen.

In substructure ii, useful values of Y, U and R³⁴, R³⁵, R³⁶, R³⁷, R³⁸, R³⁹ and R⁴⁰ are described above. Preferred compounds include those where U is hydroxy.

Compounds of Formula I include those compounds of the following structure:

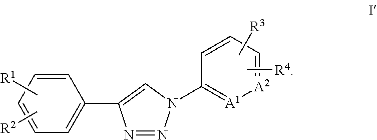

Preferred compounds of formula I′ are those where one of R¹ and R² is hydroxy, —OCO(C₁₋₄ alkyl), mono(C₁₋₄ alkyl)amino, di(C₁₋₄ alkyl)amino or —NHCO(C₁₋₄ alkyl), and the other of R¹ and R² is hydrogen. Preferred compounds of Formula I′ are those where R⁴ is hydrogen, radiohalogen or halogen. Preferred compounds of formula I′ are those where R³ is hydrogen or fragment ii. Examples of preferred compounds include:

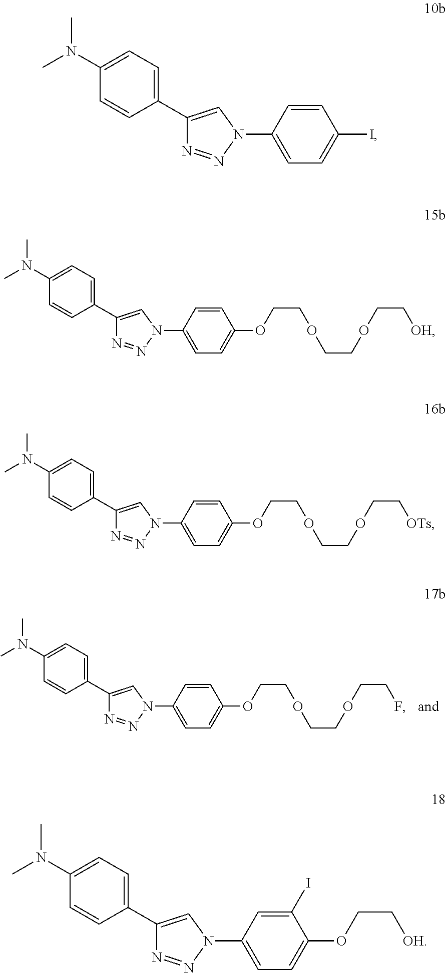

In another aspect, the present invention is directed to compounds of Formula II, having the following structure:

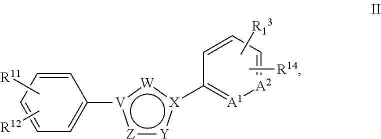

or a pharmaceutically acceptable salt thereof, wherein:
W, Y and Z are each independently CH, N, NH, O or S; provided that at least one of W, Y and Z is N or O;
V and X are independently C or N;
A¹ and A² are independently N, CR¹³ or CR¹⁴ as permitted;
R¹¹ and R¹² are independently:
   —(CH₂)ₚNR^{a}R^{b}, wherein R^{a} and R^{b} are independently hydrogen, (C₁₋₄)alkyl, hydroxy(C₁₋₄)alkyl or halo(C₁₋₄)alkyl, and p is an integer from 0 to 5; hydroxy; (C₁₋₄)alkoxy; hydroxy(C₁₋₄)alkyl; halogen; cyano; hydrogen; nitro; (C₁-C₄)alkyl; halo(C₁-C₄)alkyl; formyl; —NHCO(C₁₋₄ alkyl), o-OCO(C₁₋₄ alkyl);
R¹⁴ is hydrogen;
R¹³ is:

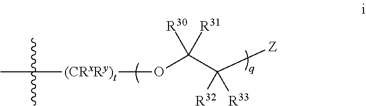

wherein q is an integer from 1 to 10, R^{x} and R^{y} are hydrogen, hydroxy or (C₁₋₄)alkyl; t is 0, 1, 2 or 3; and Z, R³⁰, R³¹, R³² and R³³ are as described; and Z is -Ch;

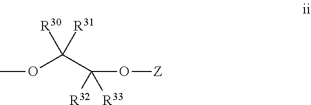

wherein Z is -Ch, R³⁰, R³¹, R³² and R³³ are as described above, and

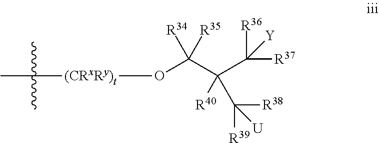

wherein R^{x} and R^{y} are hydrogen, hydroxy or (C₁₋₄)alkyl; t is 0, 1, 2 or 3; Y is -Ch; U is hydrogen, hydroxy, halogen, halogen substituted benzoyloxy, halogen substituted phenyl(C₁₋₄)alkyl, halogen substituted aryloxy, or halogen substituted (C₆₋₁₀)aryl; and R³⁴, R³⁵, R³⁶, R³⁷, R³⁸, R³⁹ and R⁴⁰ are in each instance independently hydrogen, halogen, hydroxy, (C₁₋₄)alkoxy, (C₁₋₄)alkyl, or hydroxy(C₁₋₄)alkyl;
iv. —(CH₂)_{w}—O-Ch, wherein w is an integer from 1 to 10;
v. -Ch; or
vi. —(CH₂)_{w}-Ch, wherein w is an integer from 1 to 10;
   wherein, the moiety “-Ch” is a chelating ligand capable of complexing with a metal to form a metal chelate. Many ligands are known in the art and arc suitable for use as a labeling moiety for the compounds of the present invention. Those of skill in the art will understand that such ligands provide a way to label compounds and the invention is not limited to particular ligands, many of which are interchangeable. Preferably, this ligand is a tri- or tetradentate ligand, such as N₃, N₂S, NS₂, N₄ and those of the N₂S₂ type, represented by, but not limited to, the following structure:

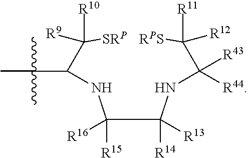

wherein R^{P} is hydrogen or a sulfhydryl protecting group, and R⁹ R¹⁰, R¹¹, R¹², R¹³, R¹⁴, R¹⁵, R¹⁶, R⁴³ and R⁴⁴ are in each instance independently hydrogen, hydroxy, amino, methylamino, dimethylamino, (C₁₋₄)alkoxy, (C₁₋₄)alkyl, and hydroxy(C₁₋₄)alkyl. When complexed with a metal such as 99m-Tc, -Ch has the following structure:

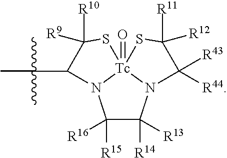

Additionally, a rhenium radioisotope can be complexed with the tetradentate ligand, rather than technetium. When the chelating moiety is not complexed with a metal, R^{P} are both hydrogen, or can be any of the variety of protecting groups available for sulfur, including methoxymethyl, methoxyethoxymethyl, p-methoxybenzyl or benzyl. Sulfur protecting groups are described in detail in Greene, T. W. and Wuts, P. G. M., Protective Groups in Organic Synthesis, 2nd Edition, John Wiley and Sons, Inc., New York (1991). Protecting group R^{P} can be removed by appropriate methods well known in the art of organic synthesis, such as trifluoroacetic acid, mercuric chloride or sodium in liquid ammonia. In the case of Lewis acid labile groups, including acetamidomethyl and benzamidomethyl, R^{P} can be left intact. Labeling of the ligand with technetium in this case will cleave the protecting group, rendering the protected diaminedithiol equivalent to the unprotected form. Further, several ligands of the general N₂S₂ type are known, and can be used interchangeably without changing the scope of the invention; and

Preferred values of R¹¹ or R¹² are hydroxy or NR^{a}R^{b}(CH₂)ₚ—, wherein p is an integer from 0 to 5, and R^{a} and R^{b} are independently hydrogen, (C₁₋₄)alkyl or (CH₂)_{d}X, where X is halogen, and d is an integer from 1 to 4. Useful values of p include integers from 0 to 5. Preferably, p is 0, 1 or 2. Most preferably, p is 0 such that R¹¹ or R¹² represents NR^{a}R^{b}. In preferred embodiments, R¹¹ is other than hydrogen and is either in the meta or para position relative to the respective bridge. A preferred value of R¹¹ is NR^{a}R^{b}, wherein R^{a} and R^{b} are independently hydrogen or (C₁₋₄)alkyl. In this embodiment, it is preferable that the (C₁₋₄)alkyl is methyl. Preferably one of R^{a} and R^{b} is hydrogen, the other is (C₁₋₄)alkyl, such as methyl or both R^{a} and R^{b} are methyl. Another preferred value of R¹¹ is hydroxy. Also preferred for R¹¹ are any groups that after administration into the body metabolize or degrade to the preferred values of R¹¹ listed above. Such groups are known in the art to constitute a prodrug and the groups capable of forming prodrugs are well-known to one of ordinary skill in the art.

Useful values of R¹³ include substructures i, ii, iii, iv, v and vi as depicted above. In preferred embodiments of Formula I, R¹³ is either in the meta or para position relative to the respective bridge. Preferably, in a compound where R¹³ is i, q is an integer from 2 to 5. Most preferably, q is 3 or 4. In substructure i, useful values of R³⁰, R³¹, R³² and R³³ independently include hydrogen, hydroxy, (C₁₋₄)alkoxy, (C₁₋₄)alkyl, and hydroxy(C₁₋₄)alkyl. Preferred compounds include those where one or more of R³⁰, R³¹, R³² and R³³ are hydrogen. More preferred compounds include those where each of R³⁰, R³¹, R³² and R³³ is hydrogen.

In substructure iii, useful values of U and R³⁴, R³⁵, R³⁶, R³⁷, R³⁸, R³⁹ and R⁴⁰ are described above. Preferred compounds include those where U is hydroxy.

Compounds of Formula I also include compounds of Formulae I″ and I′″.

In another embodiment of the present invention are compounds of Formula I″ having the following structure:

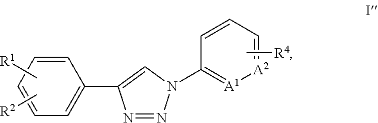

or a pharmaceutically acceptable salt thereof; wherein useful and preferred values of R¹, R², R⁴, A¹ and A² are as described under Formula I. In especially preferred compounds of Formula I″, R⁴ is a halogen or radiohalogen. More preferably, R⁴ is a radiohalogen. Non-limiting examples of compounds of Formula I″ are those that Contain a monosubstituted phenyl or heteroaryl, such as compound 10b described herein, which is monosubstituted with an iodine.

In another embodiment of the present invention arc compounds of Formula I′″ having the following structure:

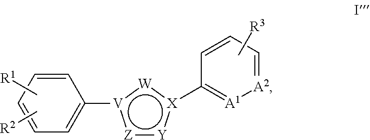

or a pharmaceutically acceptable salt thereof; wherein useful and preferred values of R¹, R², R⁴, A¹ and A² are as described under Formula I. In especially preferred compounds of Formula I′″, R³ is ii. Non-limiting examples of compounds of Formula I′″ are compounds 15b, 16b and 17b described herein.

In all embodiments of Formulae I, II and I′ containing —(CR^{x}R^{y})ₜ where t is other than zero, the compounds have the following general structure wherein there is at least one carbon-carbon bond between a substituent and the ring to which —(CR^{x}R^{y)}ₜ is bound:

Error! Objects cannot be created from editing field codes.

and for those compounds of Formula I′″ when t is other than zero:

Error! Objects cannot be created from editing field codes.

The compounds of the present invention can also contain a radioactive isotope of carbon as the radiolabel. This refers to a compound that comprises one or more radioactive carbon atoms, preferably ¹¹C, with a specific activity above that of the background level for that atom. It is well known, in this respect, that naturally occurring elements are present in the form of varying isotopes, some of which are radioactive isotopes. The radioactivity of the naturally occurring elements is a result of the natural distribution or abundance of these isotopes, and is commonly referred to as a background level. The carbon labeled compounds of the present invention have a specific activity that is higher than the natural abundance, and therefore above the background level. The composition claimed herein comprising a carbon-labeled compound(s) of the present invention will have an amount of the compound such that the composition can be used for tracing, imaging, radiotherapy, and the like.

In certain embodiments of the compounds disclosed herein, a halogen, preferably ¹⁸F, or a chelating agent is linked to the backbone through a PEG chain, having a variable number of ethoxy groups.

The compounds of Formulae I, I′, II, I″ and I′″ may also be solvated, especially hydrated. Hydration may occur during manufacturing of the compounds or compositions comprising the compounds, or the hydration may occur over time due to the hygroscopic nature of the compounds. In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

When any variable occurs more than one time in any constituent or in Formulae I, I′, II, I″ and I′″ its definition on each occurrence is independent of its definition at every other occurrence. Also combinations of substituents and/or variables are permissible only if such combinations result in stable compounds.

The term “alkyl” as employed herein by itself or as part of another group refers to both straight and branched chain radicals of up to 8 carbons, preferably 6 carbons, more preferably 4 carbons, such as methyl, ethyl, propyl, isopropyl, butyl, t-butyl, and isobutyl.

The term “alkoxy” is used herein to mean a straight or branched chain alkyl radical, as defined above, unless the chain length is limited thereto, bonded to an oxygen atom, including, but not limited to, methoxy, ethoxy, n-propoxy, isopropoxy, and the like. Preferably the alkoxy chain is 1 to 6 carbon atoms in length, more preferably 1-4 carbon atoms in length.

The term “monoalkylamine” as employed herein by itself or as part of another group refers to an amino group which is substituted with one alkyl group as defined above.

The term “dialkylamine” as employed herein by itself or as part of another group refers to an amino group which is substituted with two alkyl groups as defined above.

The term “halo” or “halogen” employed herein by itself or as part of another group refers to chlorine, bromine, fluorine or iodine and their isotopes. The term “radiohalogen” refers specifically to radioactive halogen isotopes.

The term “haloalkyl” as employed herein refers to any of the above alkyl groups substituted by one or more chlorine, bromine, fluorine or iodine with fluorine and chlorine being preferred, such as chloromethyl, iodomethyl, trifluoromethyl, 2,2,2-trifluoroethyl, and 2-chloroethyl.

The term “alkylthio” as employed herein by itself or as part of another group refers to a thioether of the structure: R—S, wherein R is a C₁₋₄ alkyl as defined above.

The term “alkylsulfonyl” as employed herein by itself or as part of another group refers to a sulfone of the structure: R—SO₂, wherein R is a C₁₋₄ alkyl as defined above.

The term “aryl” as employed herein by itself or as part of another group refers to monocyclic or bicyclic aromatic groups containing from 6 to 12 carbons in the ring portion, preferably 6-10 carbons in the ring portion, such as phenyl, naphthyl or tetrahydronaphthyl.

The term “heterocycle” or “heterocyclic ring”, as used herein except where noted, represents a stable 5- to 7-membered mono-heterocyclic ring system which may be saturated or unsaturated, and which consists of carbon atoms and from one to three heteroatoms selected from the group consisting of N, O, and S, and wherein the nitrogen and sulfur heteroatom may optionally be oxidized. Especially useful are rings contain one nitrogen combined with one oxygen or sulfur, or two nitrogen heteroatoms. Examples of such heterocyclic groups include piperidinyl, pyrrolyl, pyrrolidinyl, imidazolyl, imidazinyl, imidazolidinyl, pyridyl, pyrazinyl, pyrimidinyl, oxazolyl, oxazolidinyl, isoxazolyl, isoxazolidinyl, thiazolyl, thiazolidinyl, isothiazolyl, homopiperidinyl, homopiperazinyl, pyridazinyl, Pyrazolyl, and pyrazolidinyl, most preferably thiamorpholinyl, piperazinyl, and morpholinyl.

The term “heteroatom” is used herein to mean an oxygen atom (“O”), a sulfur atom (“S”) or a nitrogen atom (“N”). It will be recognized that when the heteroatom is nitrogen, it may form an NRR moiety, wherein the R groups independently from one another may be hydrogen or C₁₋₄ alkyl, C₂₋₄ aminoalkyl, C₁₋₄ halo alkyl, halo benzyl, or R¹ and R² are taken together to form a 5- to 7-member heterocyclic ring optionally having O, S or NR^{c} in said ring, where R^{c} is hydrogen or C₁₋₄ alkyl.

The term “heteroaryl” as employed herein refers to groups having 5 to 14 ring atoms; 6, 10 or 14Π electrons shared in a cyclic array; and containing carbon atoms and 1, 2, 3 or 4 oxygen, nitrogen or sulfur heteroatoms (where examples of heteroaryl groups are: thienyl, benzo[b]thienyl, naphtho[2,3-b]thienyl, thianthrenyl, furyl, pyranyl, isobenzofuranyl, benzoxazolyl, chromenyl, xanthenyl, phenoxathiinyl, 2H-pyrrolyl, pyrrolyl, imidazolyl, pyrazolyl, pyridyl, pyrazinyl, pyrimidinyl, pyridazinyl, indolizinyl, isoindolyl, 3H-indolyl, indolyl, indazolyl, purinyl, 4H-quinolizinyl, isoquinolyl, quinolyl, phthalazinyl, naphthyridinyl, quinazolinyl, cinnolinyl, pteridinyl, 4aH-carbazolyl, carbazolyl, α, β, or γ-carbolinyl, phenanthridinyl, acridinyl, perimidinyl, phenanthrolinyl, phenazinyl, isothiazolyl, phenothiazinyl, isoxazolyl, furazanyl and phenoxazinyl groups).

The term “aralkyl” or “arylalkyl” as employed herein by itself or as part of another group refers to C₁₋₆ alkyl groups as discussed above having an aryl substituent, such as benzyl, phenylethyl or 2-naphthylmethyl.

Preferable values under the scope of C₆₋₁₀ aryl include phenyl, naphthyl or tetrahydronaphthyl. Preferable values of under the scope of heteroaryl include thienyl, furyl, pyranyl, pyrrolyl, pyridinyl, indolyl, and imidazolyl. Preferable values under the scope of heterocycle include piperidinyl, pyrrolidinyl, and morpholinyl. A preferred embodiment of a C₆₋₁₀ aryl, heteroaryl, heterocycle, heterocycle(C₁₋₄)alkyl or C₃₋₆ cycloalkyl, contains a ring substituted with one of the following: C₁₋₄ alkylthio, C₁₋₄ alkyl sulfonyl, methoxy, hydroxy, dimethylamino or methylamino.

Another aspect of this invention is related to methods of preparing compounds of Formulae I, I′, II, I″ and I′″.

Some of the advantages of using the chemistry described herein to assemble probes targeting Aβ plaques are: 1) synthesis can be broken down to fragments and a key assembling step; 2) this reaction can be adapted to fragments with various substitution groups, therefore, facilitate the assembling of diversified groups of substituting groups; 3) the core structure (such as a tricyclic ring system of diphenyltriazole) contains three nitrogens, which reduce the lipophilicity as compared to the comparable thiophene analogs; 4) additional variation of the triazole ring, containing combinations of an assortment of nitrogen and oxygen atoms may further extend the range of five-member rings suitable for providing probes with high binding affinity; 5) various substitution groups on the tricyclic ring systems may provide a readily prepared probes to modulate the biological kinetics leading to improved signal to noise ratios by PET or SPECT imaging.

Cu(I)-catalyzed azide-alkyne cycloaddition (CuAAC) method reported by Sharpless and his collegues was utilized to assemble the iodinated ligands 10a (70%) and 10b (98%) (Scheme 1). The combination of copper(II) sulfate/sodium ascorbate was utilized in situ to prepare the copper(I) species and “click reaction” was achieved in one day at the room temperature. Subsequently, a palladium cataylzed trans-stannylation afforded the tributyltin precursors 11a and 11b for radiolabeling with 45% and 66% yields, respectively. For radioiodinated ligands, the standard iododestannylation reactions, using sodium [¹²⁵I]iodide, hydrogen peroxide and hydrochloric acid, were successfully applied to yield [¹²⁵I]10a and [¹²⁵I]10b with excellent yields (80-85%) and greater than 95% radiochemical purities.

A one-pot two-step approach reported by Fokin et.al. was utilized to synthesize the fluoro- and hydroxy pegylated diphenyltriazole derivatives 13a-b (Scheme 2). By directly reacting alkyated iodobenzene 12a-b, terminal alkyne 8a-b and sodium azide in a_one pot reaction, the desired diphenyltriazoles 13a-b were synthesized with acceptable yields (62% and 65%). Furthermore, by reacting 13b with diethylaminosulfur trifluoride (DAST) under 0° C. over 0.5 h, the fluoropegylated ligand 13c was obtained in 20% yield.

The desired diphenyltriazole derivatives 15a-b were prepared by a modified one-pot two-step approach, which was developed by Liang et al. In this reaction a chelating ligand trans-N,N′-dimethyl-1,2-cyclohexanediamine, copper(I) iodide and an equal amount of sodium ascorbate were used as the catalyts, the desired reactions were accomplished at room temperature in 3 h, the yields of reactions were 99% and 92%, respectively. The alcohols 15a-b were then converted to the tosylates 17a-b (96% and 90%), a microwave-assisted fluorination reaction afforded final products 17a-b (80% and 99%).

To obtain [¹⁸F]17a and [¹⁸F]17b, the corresponding tos.ylated precursors 16a and 16b were reacted with [¹⁸F]fluoride in the presence of Kryptofix 222 and potassium carbonate in DMSO at 120° C. for 4 min. The resulting ¹⁸F labeled crude products were purified by HPLC. The desired products were obtained in 70 min and the specific activities at the end of synthesis were 600 and 800 mCi/μmol for [¹⁸F]17a and [¹⁸F]17b, respectively. The radiochemical purities were >99% for both ligands and the radiochemical yields were 50% for [¹⁸F]17a and 30% for [¹⁸F]17b (decay corrected).

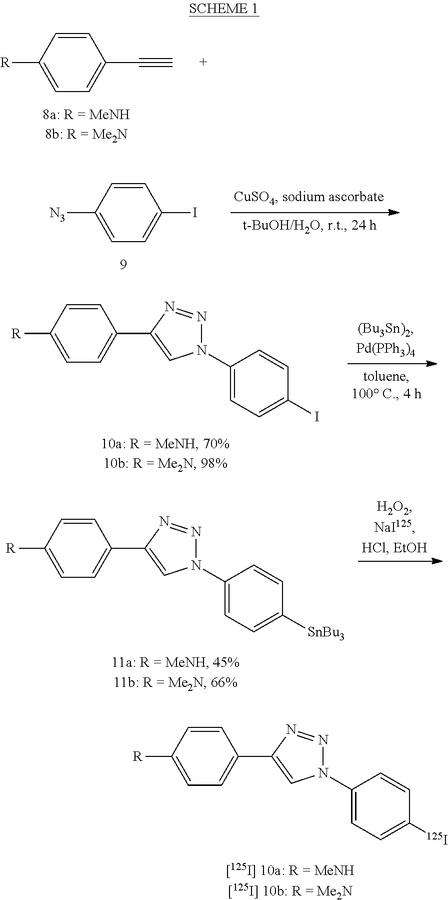

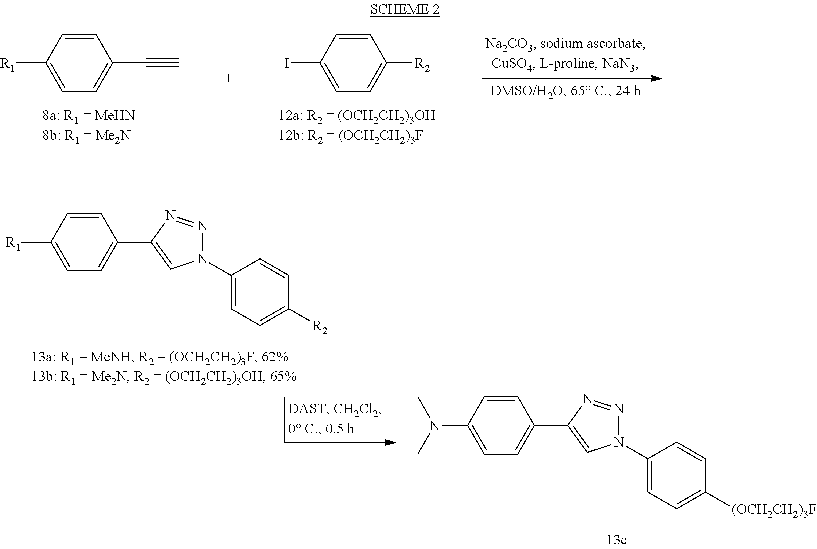

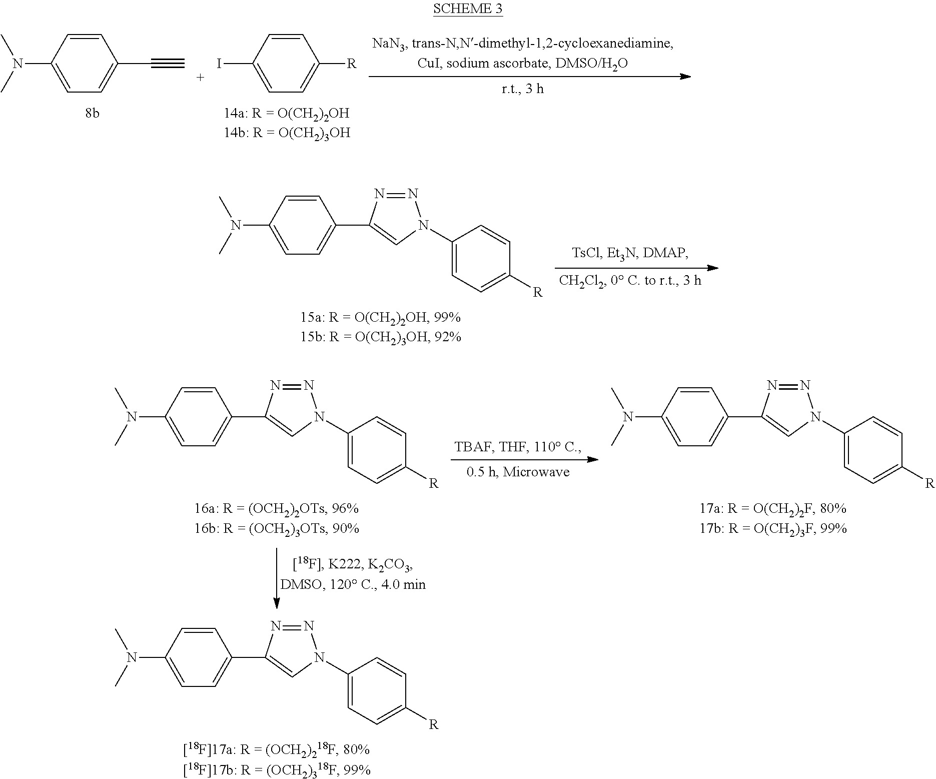

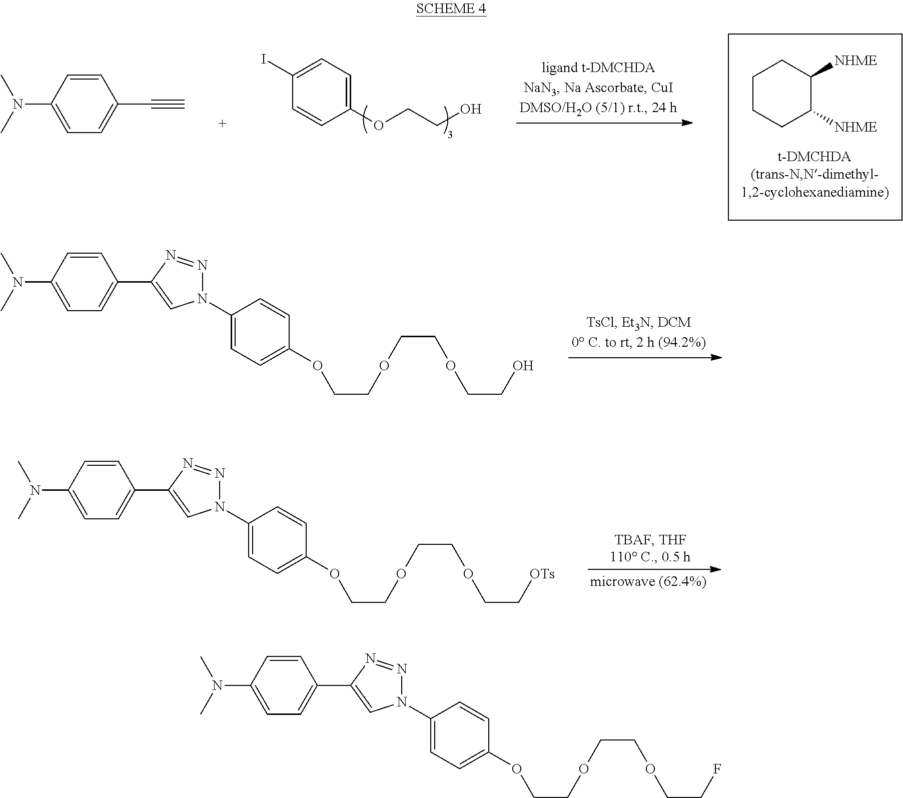

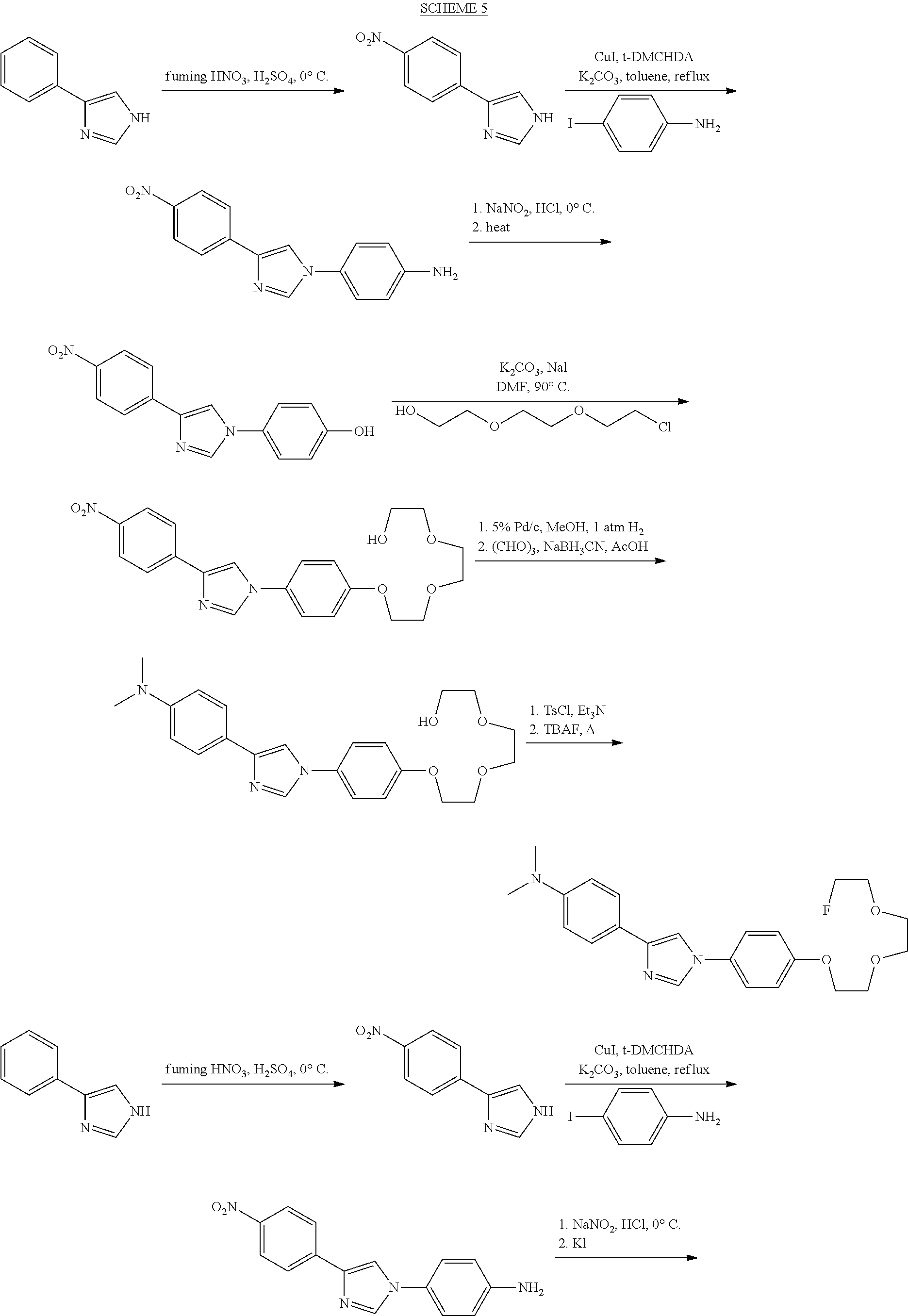

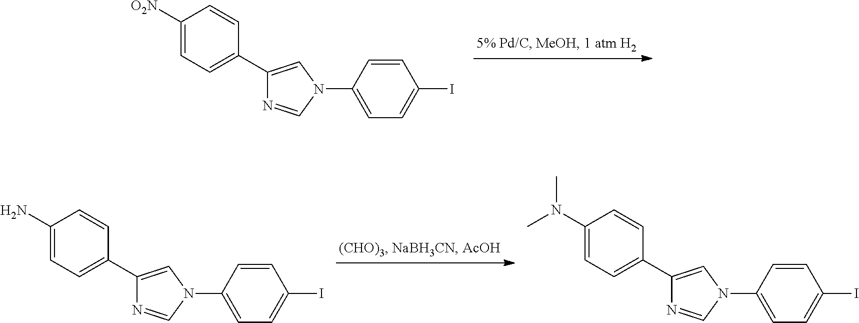

Other heteroaromatic compounds are synthesized according to the following schemes 6-20.

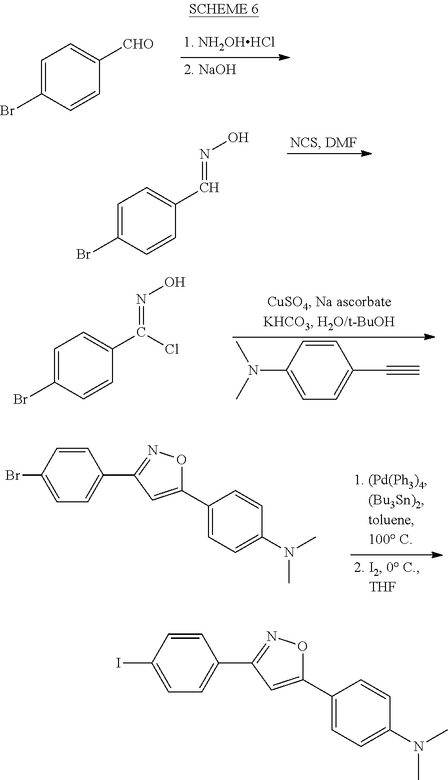

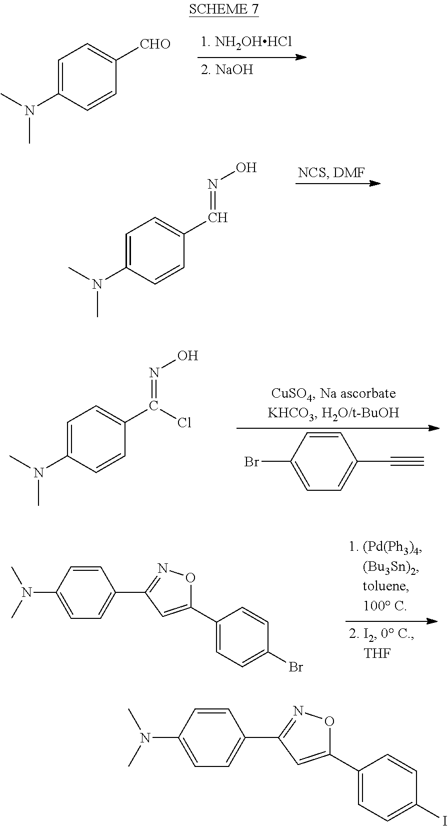

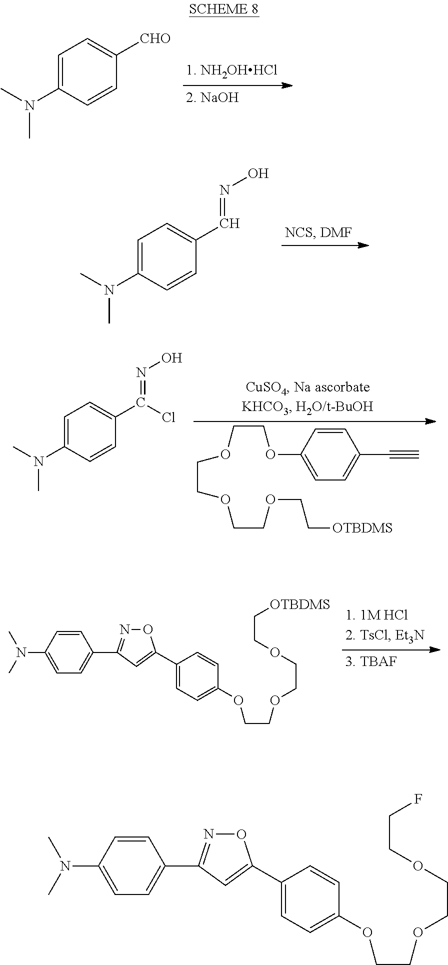

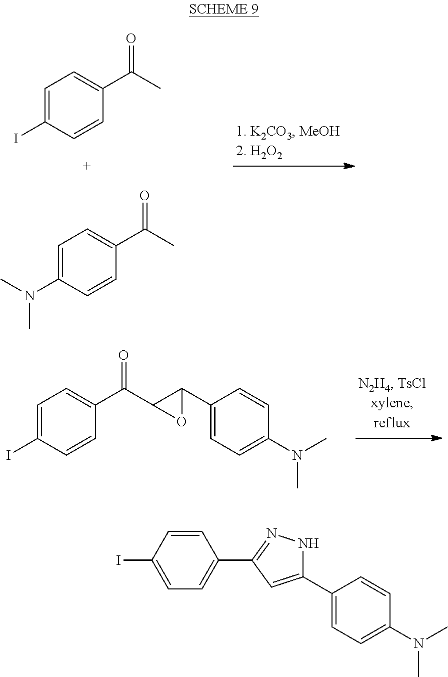

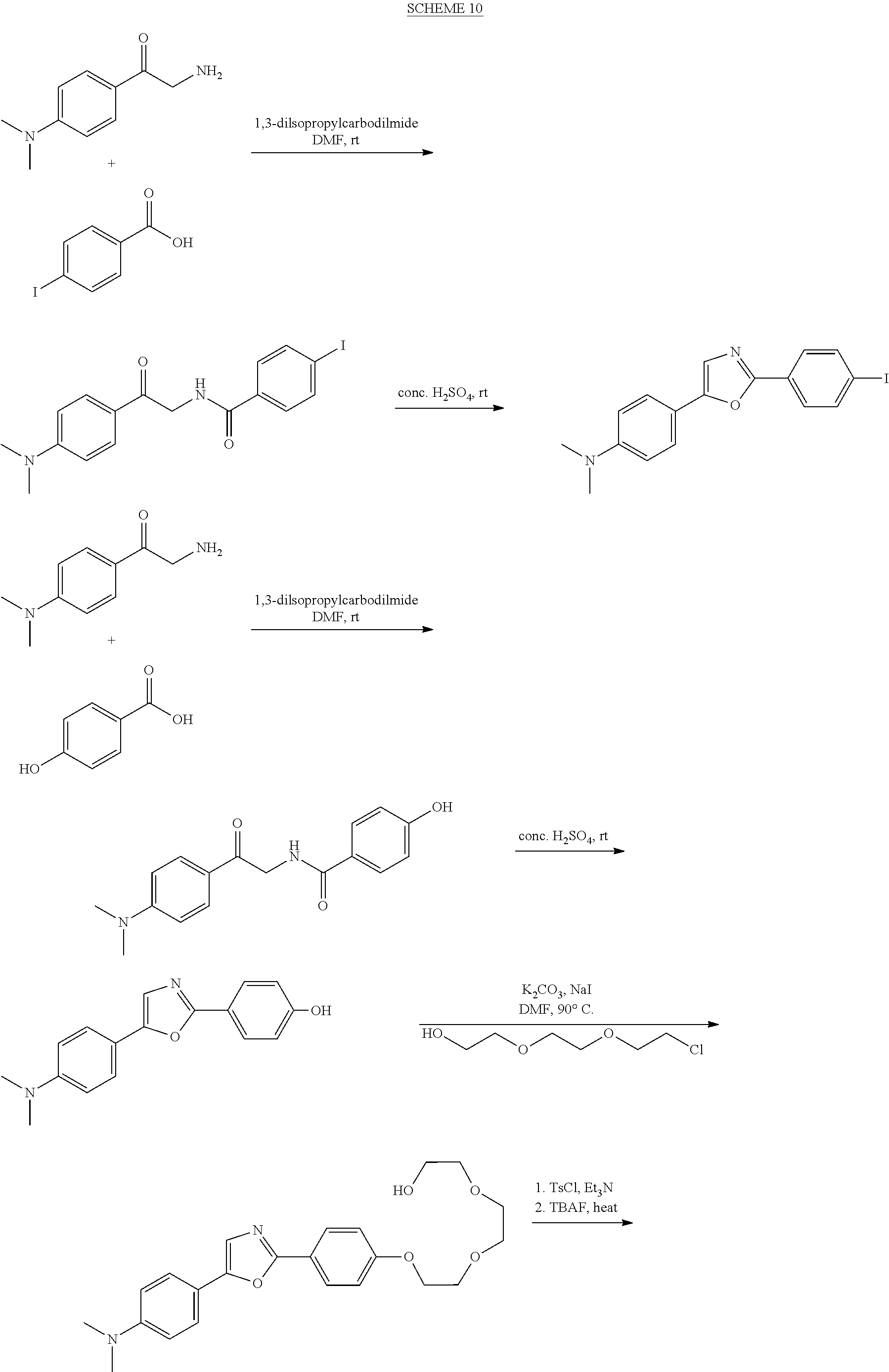

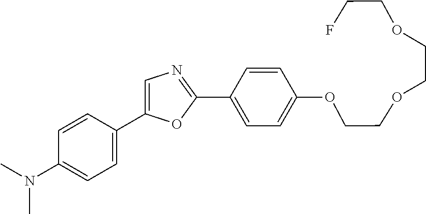

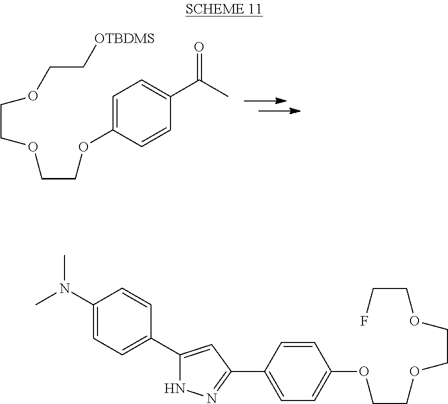

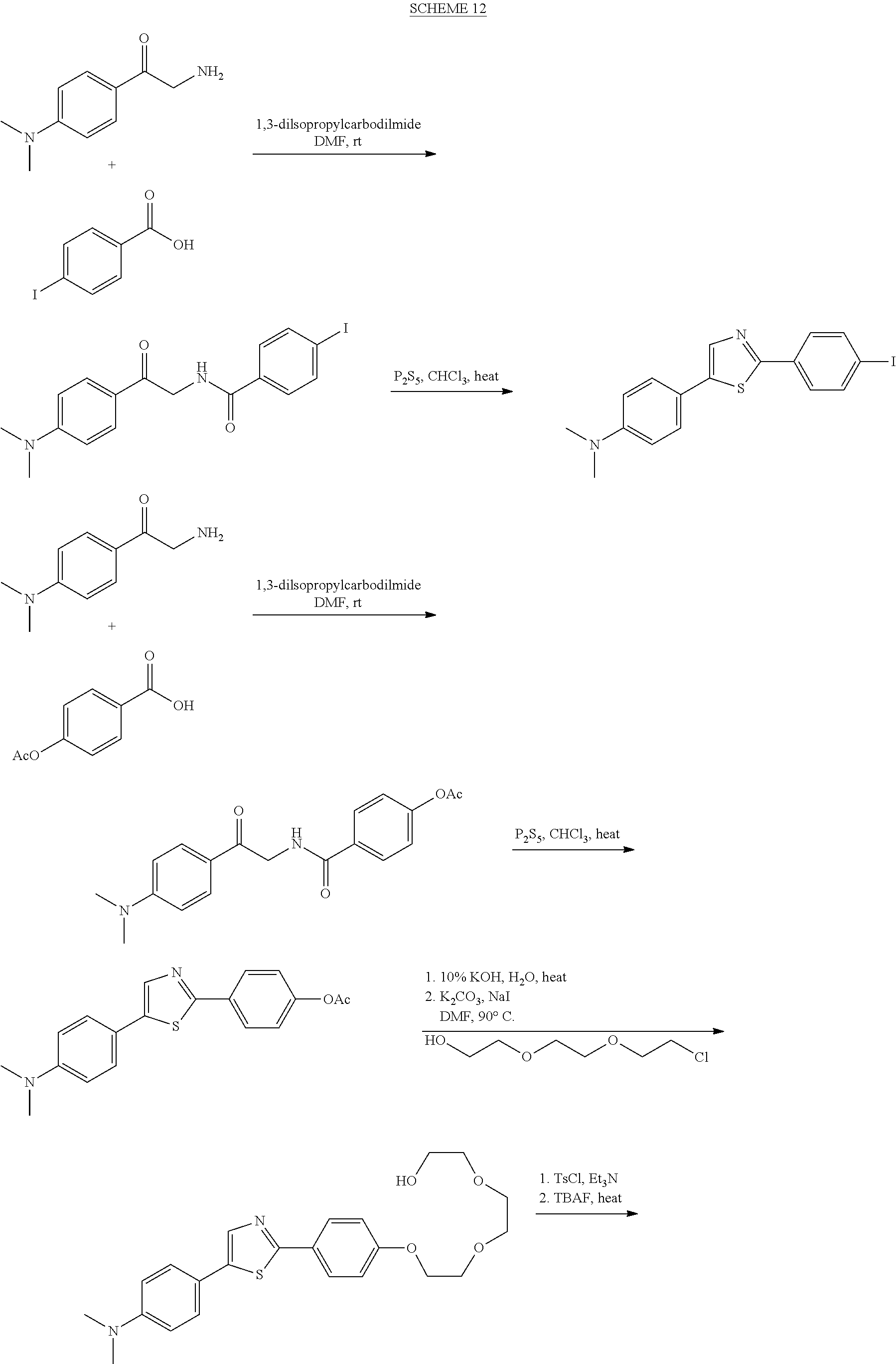

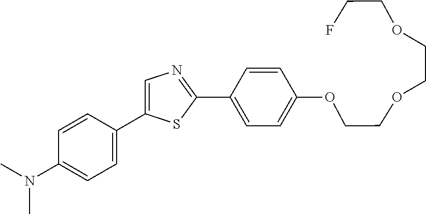

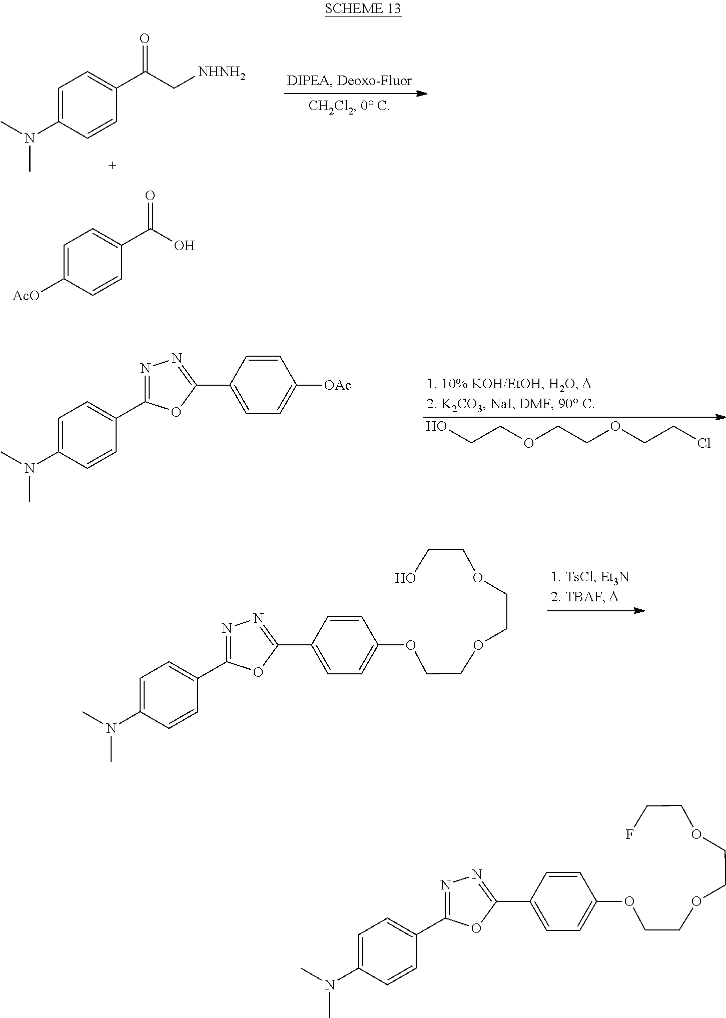

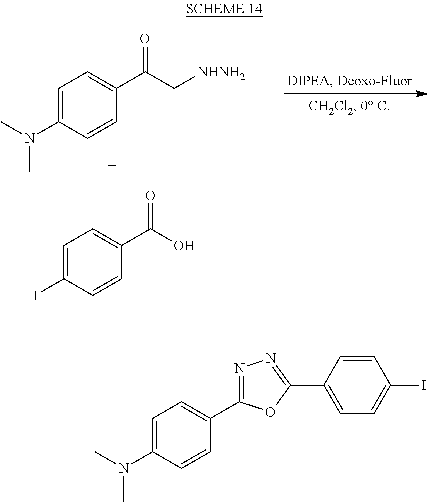

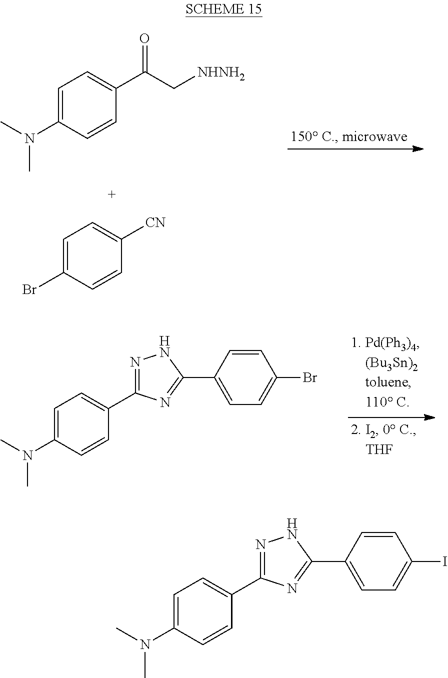

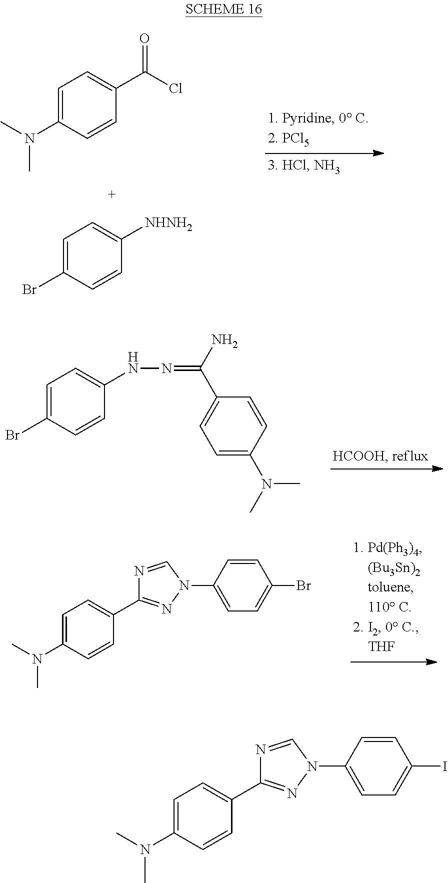

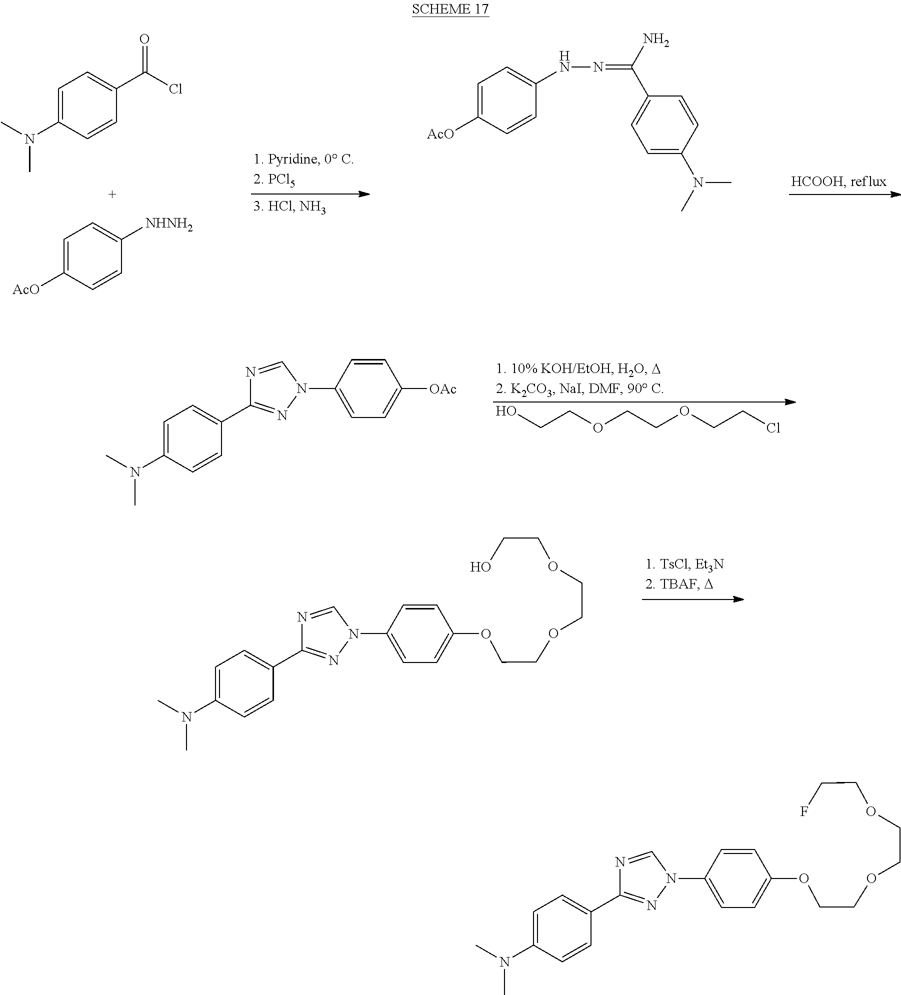

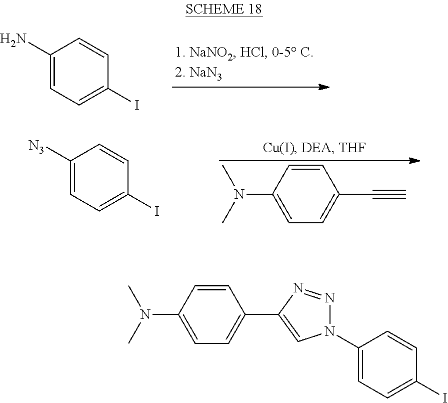

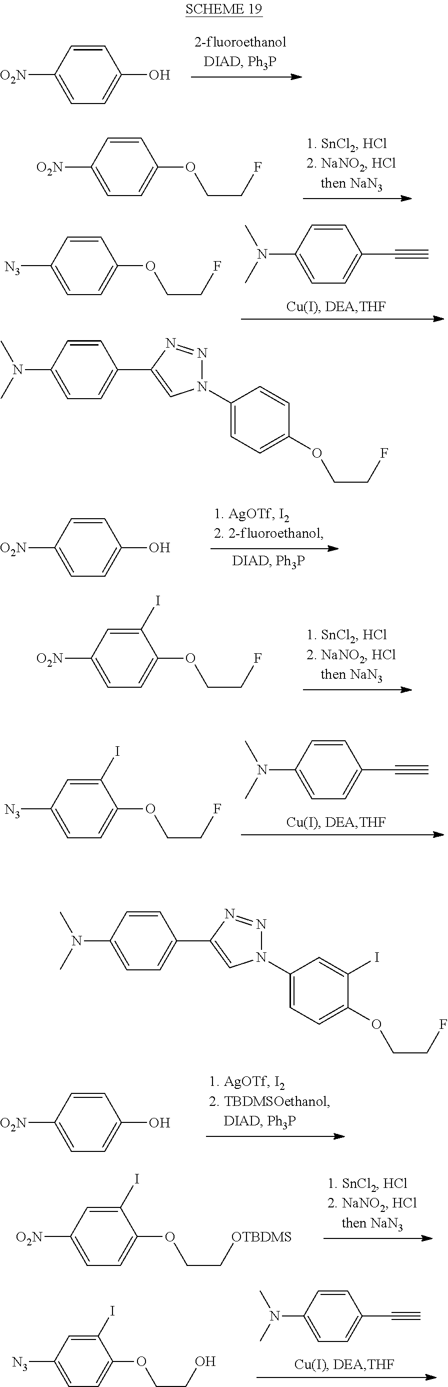

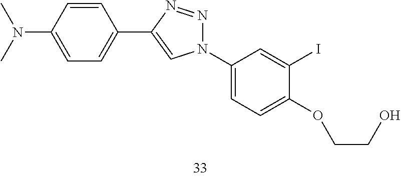

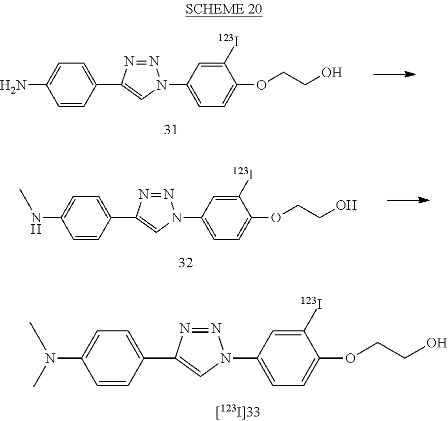

Tc-99m complexes can be prepared as follows. A small amount of non-radiolabeled compound (1-2 mg) is dissolved in 100 μL EtOH and mixed with 200 μL HCl (1 N) and 1 mL Sn-glucoheptonate solution (containing 8-32 μg SnCl₂ and 80-320 μg Na-glucoheptonate, pH 6.67) and 50 μL EDTA solution (0.1 N). [^{99m}Tc]Pertechnetatc (100-200 μL; ranging from 2-20 mCi) saline solution are then added. The reaction is heated for 30 min at 100° C., then cooled to room temperature. The reaction mixture is analyzed on TLC (EtOH:conc. NH₃ 9:1) for product formation and purity check. The mixture can be neutralized with phosphate buffer to pH 5.0.

The present invention further relates to a method of preparing a technetium-99m complex according to the present invention by reacting technetium-99m in the form of a pertechnetate in the presence of a reducing agent and optionally a suitable chelator with an appropriate Ch-containing compound.

The reducing agent serves to reduce the Tc-99m pertechnetate which is eluted from a molybdenum-technetium generator in a physiological saline solution. Suitable reducing agents arc, for example, dithionite, formamidine sulphinic acid, diaminoethane disulphinate or suitable metallic reducing agents such as Sn(II), Fe(II), Cu(I), Ti(III) or Sb(III). Sn(II) has proven to be particularly suitable.

For the above-mentioned complex-forming reaction, technetium-99m is reacted with an appropriate compound of the invention as a salt or in the form of technetium bound to comparatively weak chelators. In the latter case the desired technetium-99m complex is formed by ligand exchange. Examples of suitable chelators for the radionuclide are dicarboxylic acids, such as oxalic acid, malonic acid, succinic acid, maleic acid, orthophtalic acid, malic acid, lactic acid, tartaric acid, citric acid, ascorbic acid, salicylic acid or derivatives of these acids; phosphorus compounds such as pyrophosphates; or enolates. Citric acid, tartaric acid, ascorbic acid, glucoheptonic acid or a derivative thereof are particularly suitable chelators for this purpose, because a chelate of technetium-99m with one of these chelators undergoes the desired ligand exchange particularly easily.

The most commonly used procedure for preparing [Tc^{v}O]⁻³N₂S₂ complexes is based on stannous (II) chloride reduction of [^{99m}Tc]pertechnetate, the common starting material. The labeling procedure normally relies on a Tc-99m ligand exchange reaction between Tc-99m (Sn)-glucoheptonate and the N₂S₂ ligand. Preparation of stannous (II) chloride and preserving it in a consistent stannous (II) form is critically important for the'success of the labeling reaction. To stabilize the air-sensitive stannous ion it is a common practice in nuclear medicine to use a lyophilized kit, in which the stannous ion is in a lyophilized powder form mixed with an excess amount of glucoheptonate under an inert gas like nitrogen or argon. The preparation of the lyophilized stannous chloride/sodium glucoheptonate kits ensures that the labeling reaction is reproducible and predictable. The N₂S₂ ligands are usually air-sensitive (thiols are easily oxidized by air) and there are subsequent reactions which lead todecomposition of the ligands. The most convenient and predictable method to preserve the ligands is to produce lyophilized kits containing 100-500 μg of the ligands under argon or nitrogen.

When the compounds of this invention are to be used as imaging agents, they must be labeled with suitable radioactive halogen isotopes. Although ¹²⁵I-isotopes are useful for laboratory testing, they will generally not be useful for actual diagnostic purposes because of the relatively long half-life (60 days) and low gamma-emission (30-65 Key) of ¹²⁵I. The isotope ¹²³I has a half life of thirteen hours and gamma energy of 159 KeV, and it is therefore expected that labeling of ligands to be used for diagnostic purposes would be with this isotope. Other isotopes which may be used include ¹³¹I (half life of 2 hours). Suitable bromine isotopes include ⁷⁷Br and ⁷⁶Br.

The radiohalogenated compounds of this invention lend themselves easily to formation from materials which could be provided to users in kits. Kits for forming the imaging agents can contain, for example, a vial containing a physiologically suitable solution of an intermediate of Formulae I, I′, II, I″ and I′″ in a concentration and at a pH suitable for optimal complexing conditions. The user would add to the vial an appropriate quantity of the radioisotope, e.g., Na¹²³I, and an oxidant, such as hydrogen peroxide. The resulting labeled ligand may then be administered intravenously to a patient, and receptors in the brain imaged by means of measuring the gamma ray or photo emissions therefrom.

Since the radiopharmaceutical composition according to the present invention can be prepared easily and simply, the preparation can be carried out readily by the user. Therefore, the present invention also relates to a kit, comprising:
(1) A non-radiolabeled compound of the invention, the compound optionally being in a dry condition; and also optionally having an inert, pharmaceutically acceptable carrier and/or auxiliary substances added thereto; and
(2) a reducing agent and optionally a chelator;

wherein ingredients (1) and (2) may optionally be combined; and
further wherein instructions for use with a prescription for carrying out the above-described method by reacting ingredients (1) and (2) with technetium-99m in the form of a pertechnetate solution may be optionally included.

Examples of suitable reducing agents and chelators for the above kit have been listed above. The pertechnetate solution can be obtained by the user from a molybdenum-technetium generator. Such generators are available in a number of institutions that perform radiodiagnostic procedures. As noted above the ingredients (1) and (2) may be combined, provided they are compatible. Such a monocomponent kit, in which the combined ingredients are preferably lyophilized, is excellently suitable to be reacted by the user with the pertechnetate solution in a simple manner.

When desired, the radioactive diagnostic agent may contain any additive such as pH controlling agents (e.g., acids, bases, buffers), stabilizers (e.g., ascorbic acid) or isotonizing agents (e.g., sodium chloride).

The term “pharmaceutically acceptable salt” as used herein refers to those carboxylate salts or acid addition salts of the compounds of the present invention which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the invention. The term “salts” refers to the relatively nontoxic, inorganic and organic acid addition salts of compounds of the present invention. Also included are those salts derived from non-toxic organic acids such as aliphatic mono and dicarboxylic acids, for example acetic acid, phenyl-substituted alkanoic acids, hydroxy alkanoic and alkanedioic acids, aromatic acids, and aliphatic and aromatic sulfonic acids. These salts can be prepared in situ during the final isolation and purification of the compounds or by separately reacting the purified compound in its free base form with a suitable organic or inorganic acid and isolating the salt thus formed. Further representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactiobionate and laurylsulphonate salts, propionate, pivalate, cyclamate, isethionate, and the like. These may include cations based on the alkali and alkaline earth metals, such as sodium, lithium, potassium, calcium, magnesium, and the like, as well as, nontoxic ammonium, quaternary ammonium and amine cations including, but not limited to ammonium, tetramethylammonium, tetraethylammonium, methylamine, dimethylamine, trimethylamine, triethylamine, ethylamine, and the like. (See, for example, Berge S. M., et al., Pharmaceutical Salts, J. Pharm. Sci. 66:1-19 (1977) which is incorporated herein by reference.)

In the first step of the present method of imaging, a labeled compound of Formulae I, I′, II, I″ and I′″ is introduced into a tissue or a patient in a detectable quantity. The compound is typically part of a pharmaceutical composition and is administered to the tissue or the patient by methods well known to those skilled in the art.

The administration of the labeled compound to a patient can be by a general or local administration route. For example, the compound can be administered either orally, rectally, parenterally (intravenous, by intramuscularly or subcutaneously), intracisternally, intravaginally, intraperitoneally, intravesically, locally (powders, ointments or drops), or as a buccal or nasal spray. The labeled compound may be administered to the patient such that it is delivered throughout the body. Alternatively, the labeled compound can be administered to a specific organ or tissue of interest. For example, it is desirable to locate and quantitate amyloid deposits in the brain in order to diagnose or track the progress of Alzheimer's disease in a patient. One of the most desirable characteristics of an in vivo imaging agent of the brain is the ability to cross the intact blood-brain barrier after a bolus iv injection.

In a preferred embodiment of the invention, the labeled compound is introduced into a patient in a detectable quantity and after sufficient time has passed for the compound to become associated with amyloid deposits, the labeled compound is detected noninvasively inside the patient. In another embodiment of the invention, a radiolabeled compound of Formula I, I′, II, I″ or I′″ is introduced into a patient, sufficient time is allowed for the compound to become associated with amyloid deposits, and then a sample of tissue from the patient is removed and the labeled compound in the tissue is detected apart from the patient. In a third embodiment of the invention, a tissue sample is removed from a patient and a labeled compound of Formula I, I′, II, I″ or I′″ is introduced into the tissue sample. After a sufficient amount of time for the compound to become bound to amyloid deposits, the compound is detected.

The term “tissue” means a part of a patient's body. Examples of tissues include the brain, heart, liver, blood vessels, and arteries. A detectable quantity is a quantity of labeled compound necessary to be detected by the detection method chosen. The amount of a labeled compound to be introduced into a patient in order to provide for detection can readily be determined by those skilled in the art. For example, increasing amounts of the labeled compound can be given to a patient until the compound is detected by the detection method of choice. A label is introduced into the compounds to provide for detection of the compounds.

The term “patient” means humans and other animals. Those skilled in the art are also familiar with determining the amount of time sufficient for a compound to become associated with amyloid deposits. The amount of time necessary can easily be determined by introducing a detectable amount of a labeled compound of Formula I, I′, II, I″ or I′″ into a patient and then detecting the labeled compound at various times after administration.

The term “associated” means a chemical interaction between the labeled compound and the amyloid deposit. Examples of associations include covalent bonds, ionic bonds, hydrophilic-hydrophilic interactions, hydrophobic-hydrophobic interactions, and complexes.

Those skilled in the art are familiar with the various ways to detect labeled compounds. For example, magnetic resonance imaging (MRI), positron emission tomography (PET), or single photon emission computed tomography (SPECT) can be used to detect radiolabeled compounds. The label that is introduced into the compound will depend on the detection method desired. For example, if PET is selected as a detection method, the compound must possess a positron-emitting atom, such as ¹¹C or ¹⁸F.

The radioactive diagnostic agent should have sufficient radioactivity and radioactivity concentration which can assure reliable diagnosis. For instance, in case of the radioactive metal being technetium-99m, it may be included usually in an amount of 0.1 to 50 mCi in about 0.5 to 5.0 ml at the time of administration. The amount of a compound of Formulae I, I′, II, I″ or I′″ may be such as sufficient to form a stable chelate compound with the radioactive metal.

The thus formed chelate compound as a radioactive diagnostic agent is sufficiently stable, and therefore it may be immediately administered as such or stored until its use. When desired, the radioactive diagnostic agent may contain any additive such as pH controlling agents (e.g., acids, bases, buffers), stabilizers (e.g., ascorbic acid) or isotonizing agents (e.g., sodium chloride).

The imaging of amyloid deposits can also be carried out quantitatively so that the amount of amyloid deposits can be determined.

Preferred compounds for imaging include a radioisotope such as ¹¹C, ¹²³I, ¹²⁵I, ¹³¹I, ¹⁸F, ⁷⁶Br or ⁷⁷Br.

Another aspect of the invention is a method of inhibiting amyloid plaque aggregation. The present invention also provides a method of inhibiting the aggregation of amyloid proteins to form amyloid deposits, by administering to a patient an amyloid inhibiting amount of a compound of the above Formulae I, I′, II, I″ or I′″.

Those skilled in the art are readily able to determine an amyloid inhibiting amount by simply administering a compound of Formulae I, I′, II, I″ or I′″ to a patient in increasing amounts until the growth of amyloid deposits is decreased or stopped. The rate of growth can be assessed using imaging as described above or by taking a tissue sample from a patient and observing the amyloid deposits therein. The compounds of the present invention can be administered to a patient at dosage levels in the range of about 0.1 to about 1,000 mg per day. For a normal human adult having a body weight of about 70 kg, a dosage in the range of about 0.01 to about 100 mg per kilogram of body weight per day is sufficient. The specific dosage used, however, can vary. For example, the dosage can depend on a number of factors including the requirements of the patient, the severity of the condition being treated, and the pharmacological activity of the compound being used. The determination of optimum dosages for a particular patient is well known to those skilled in the art.

The following examples are illustrative, but not limiting, of the method and compositions of the present invention. Other suitable modifications and adaptations of the variety of conditions and parameters normally encountered and obvious to those skilled in the art are within the spirit and scope of the invention.

All reagents used were commercial products and were used without further purification unless otherwise indicated. Flash chromatography (FC) was performed using silica gel 60 (230-400 mesh, Sigma-Aldrich). Preparative thin layer chromatography (PTLC) was performed on silica gel plates with a fluorescent indicator that was visualized with light at 254 nm (Analtech). For each procedure, “standard workup” refers to the following steps: addition of the indicated organic solvent, washing the organic layer with water then brine, separation of the organic layer from the aqueous layer, drying off the combined organic layers with sodium sulfate or magnesium sulfate, filtering off the solid and concentrating the filtrate under reduced pressure. ¹H NMR spectra were obtained at 200 MHz and ¹³C NMR spectra were recorded at 50 MHz (Bruker DPX spectrometer). Chemical shifts were reported as δvalues (parts per million) relative to internal TMS. Coupling constants were reported in hertz. The multiplicity is defined by s (singlet), d (doublet), t (triplet), br (broad) or m (multiplet). High-resolution MS experiments were performed at the McMaster Regional Centre for Mass Spectrometry using a Micromass/Waters GCT instrument (GC-EI/CI Time of Flight Mass Spectrometer). Two systems were used to confirm the purity of some compounds listed in this section, system A conditions: Hamilton PRP-1 reverse-phased analytical column (4.1×250 mm, 10 μm), 80/20 CH₃CN/1 mM ammonium formate (pH=7) water buffer, 1.0 mL/min, UV 350 nm; system B conditions: Phenomenex Silica column (4.6×250 mm, 5 μm), 40/60 EtOAc/Hexanes, 1.0 mL/min, UV 350 nm. All compounds reported in this paper showed greater than 95% purity in both systems.

### EXAMPLES

### Example 1

### Synthesis of Preferred Compounds

### 4-(1-(4-iodophenyl)-1H-1,2,3-triazol-4-yl)-N-methylbenzenamine (10a)

Alkyne 8a (0.042 g, 0.32 mmol), azide 9 (0.32 mmol, 0.079 g) and sodium ascorbate (0.16 mL, fresh prepared 1.0 M solution) were added into tert-butanol/H₂O (1/1, 2 mL) and the whole mixture was degassed with nitrogen for 10 min. Copper(II) sulfate (CuSO₄, 1.0 M aqueous solution, 16 μL) was added and the reaction mixture was vigorously stirred at room temperature (r.t.) for 24 h. After diluted with ice-cold water (10 mL), the mixture was filtered and washed with cold water and ice-cold Et₂O. The solid was dried under vacuum to afford 10a (0.084 g, 70%) as pale green solid. ¹H NMR ((CD₃)₂CO) δ8.75 (s, 1H), 7.99 (d, 2H, J=8.8 Hz), 7.82-7.72 (m, 4H), 6.69 (d, 2H, J=8.7 Hz), 5.19 (brs, 1H), 2.85, 2.83 (s, s, 3H, —NCH₃). ¹³C NMR (DMSOd₆) δ 150.0, 148.4, 138.5, 136.4, 126.4, 121.6, 117.3, 117.0, 111.7, 93.7, 29.6. HRMS calcd for C₁₅H₁₃IN₄ (M⁺), 376.0185; found, 376.0168.

### 4-(1-(4-iodophenyl)-1H-1,2,3-triazol-4-yl)-N,N-dimethylbenzenamine (10b)

Following the procedure in the preparation of 10a, compound 10b was prepared from alkyne 8b (0.073 g, 0.50 mmol) and 9 (0.147 g, 0.60 mmol) as a pale yellow solid (0.191 g, 98% yield). ¹H NMR ((CD₃)₂CO) δ8.81 (s, 1H), 7.99 (d, 2H, J=8.9 Hz), 7.86-7.73 (m, 4H), 6.84 (d, 2H, J=8.9 Hz), 3.00 (s, 6H). ¹³C NMR (DMSO-d₆). 8.81 (s, 1H), 7.99 (d, 2H, J=8.9 Hz), 7.86-7.73 (m, 4H), 6.84 (d, 2H, J=8.9 Hz), 3.00 (s, 6H). HRMS calcd for C₁₆H₁₅IN₄ (M⁺), 390.0341; found, 390.0332.

### 4-(1-(4-(2-(2-(2-fluoroethoxy)ethoxy)ethoxy)phenyl)-1H-1,2,3-triazol-4-yl)-N-methylbenzenamine (13a)

To a 20 mL scintilation vial was added alkyne 8a (0.040 g, 0.3 mmol), iodobenzene 12b (0.106 g, 0.3 mmol), Na₂CO₃ (0.003 g, 0.03 mmol), L-proline (0.0035 g, 0.003 mmol), NaN_{3 (}0.029 g, 0.45 mmol), sodium ascorbate (0.006 g, 0.03 mmol), CuSO₄ (1.0 M aqueous solution, 0.015 mL) and 1.0 ml, mixed solvent of DMSO and H₂O (9/1, V/V). The reaction mixture was purged by nitrogen 10 min for deoxygen and then was heated to 65° C. for 24 h. After cooling down to r.t., the reaction mixture was poured into diluted ammonia (20 mL) and extracted with EtOAc (3×15 mL). The combined organic phase was washed with brine (2×10 mL), dried over Na₂SO₄, filtered and concentrated. The residue was submitted to FC (EtOAc/Hexanes, 70/30) to afford a light brown solid 13a (0.745 g, 62%). ¹H NMR (CDCl₃) δ7.97 (s, 1H), 7.76-7.63 (m, 4H), 7.06 (dt, 2H, J₁=9.0 Hz, J₂=2.7 Hz), 6.72(d, 2H, J=8.6 Hz), 4.72-4.68 (m, 1H), 4.49-4.44 (m, 1H), 4.20 (t, 2H, J=2.5 Hz), 3.94-3.68 (m, 8H), 2.90 (s, 3H). ¹³C NMR (CDCl₃) δ 159.0, 149.6, 148.9, 131.0, 127.1, 122.1, 119.5, 116.4, 115.6, 112.6, 85.0, 81.6, 71.04, 71.00, 70.8, 70.4, 69.8, 68.0, 30.7. HRMS calcd for C₂₁H₂₅FN₄O₃ (M⁺), 400.1911; found, 400.1895.

### 2-(2-(2-(4-(4-(4-(dimethylaminophenyl)-1H-1,2,3-triazol-1-yl)phenoxy)ethoxy)ethoxy)ethanol (13b)

Following the procedure in the preparation of 13a, compound 13b was prepared from 12a (0.176 g, 0.5 mmol) as a light yellow solid (0.135 g, 65% yield). ¹H NMR (CDCl₃) δ7.97 (s, 1H), 7.76 (d, 2H, J=8.7 Hz), 7.64 (d, 2H, J=8.9 Hz), 7.02 (d, 2H, J=8.9 Hz), 6.79(d, 2H, J=8.6 Hz), 4.17 (t, 2H, J=4.6 Hz), 3.85 (t, 2H, J=4.6 Hz), 3.72-3.60 (m, 8H), 2.99 (s, 6H), 2.61 (brs, 1H). ¹³C NMR (CDCl₃) δ159.0, 150.5, 148.8, 131.0, 129.4, 126.9, 122.1, 116.5, 115.6, 112.8, 72.6, 71.0, 70.5, 69.8, 68.0, 61.9, 40.7. HRMS calcd for C₂₂H₂₈N₄O₄ (M⁺), 412.2111; found, 412.2106.

### 4-(1-(4-(2-(2-(2-fluoroethoxy)ethoxy)ethoxy)phenyl)-1H-1,2,3-triazol-4-yl)-N,N-dimethylbenzenamine (13c)

To a stirred solution of 13b (0.062 g, 0.15 mmol) in CH₂Cl₂ (5 mL) cooled with ice bath (0° C.) was added diethylaminosulfur trifluoride (DAST, 0.039 mL, 0.30 mmol) dropwisely. After addition, the reaction mixture was maintained at 0° C. for 0.5 h and was submitted to PTLC (EtOAc/Hexanes, 70/30) to provide a light brown solid as 13c (0.013 g, 21%). ¹H NMR (CDCl₃) δ7.99 (s, 1H), 7.79(d, 2H, J=8.7 Hz), 7.68 (d, 2H, J=9.0 Hz), 7.06 (d, 2H, J=9.0 Hz), 6.86 (d, 2H, J=8.1 Hz), 4.70 (t, 1H, J=4.2 Hz), 4.46 (t, 1H, J=4.2 Hz), 4.21 (t, 2H, J=4.7 Hz), 3.93-3.68 (m, 8H), 3.02 (s, 6H). ¹³C NMR (CDCl₃) δ159.2, 148.8, 131.1, 127.1, 122.2, 116.6, 115.7, 113.4, 85.0, 81.7, 71.2, 71.1, 70.9, 70.5, 70.0, 69.8, 68.1, 41.1. HRMS calcd for C₂₂H₂₇FN₄O₃ (M⁺), 414.2067; found, 414.2067.

### 2-(4-(4-(4-(dimethylaminophenyl)-1H-1,2,3-triazol-1-yl)phenoxy)ethanol (15a)

To a 15 mL two-neck flask was added alkyne 8b (0.145 g, 1.0 mmol), iodobenzene 14a (0.264 g, 1.0 mmol)), trans-N,N′-dimethyl-1,2-cyclohexanediamine (0.024 mL, 0.15 mmol), NaN₃ (0.072 g, 1.1 mmol), sodium ascorbate (0.02 g, 0.10 mmol), CuI (0.019 g, 0.10 mmol) and 3 mL mixed solvent of DMSO and H₂O (5/1, V/V). The reaction mixture was purged by nitrogen 10 min for deoxygen and then was vigorously stirred at r.t. for 3 h. After diluted with ice-cold water (15 mL), the mixture was filtered and washed with ice-cold water and ice-cold Et₂O. The solid was dried to afford a pale yellow solid 15a (0.323 g, 98% yield). ¹H NMR ((CD₃)₂CO) δ8.64 (s, 1H), 7.87-7.76 (m, 4H), 7.16 (dt, 2H, J₁=9.1 Hz, J₂=2.8 Hz), 6.83 (dt, 2H, J₁=9.0 Hz, J₂=2.1 Hz), 4.17 (t, 2H, J=4.8 Hz), 4.03-3.88 (m, 3H, —OH, —CH₂), 3.00 (s, 6H). ¹³C NMR ((CD₃)₂CO) δ160.1, 151.6, 149.4, 131.9, 127.4, 122.5, 120.0, 117.6, 116.4, 113.4, 71.2, 61.4, 61.3, 40.6. HRMS calcd for C₁₈H₂₀N₄O₂ (M⁺), 324.1586; found, 324.1583.

### 3-(4-(4-(4-(dimethylaminophenyl)-1H-1,2,3-triazol-1-yl)phenoxy)propan-1-ol (15b)

Following the procedure in the preparation of 15a, compound 15b was prepared from iodobenzene 14b (0.278 g, 1.0 mmol) as a pale yellow solid (0.310 g, 92% yield). ¹H NMR ((CD₃)₂CO) δ8.64 (s, 1H), 7.85-7.78 (m, 4H), 7.15 (dt, 2H, J₁=9.0 Hz, J₂=2.7 Hz), 6.83 (d, 2H, J=8.9 Hz), 4.20 (t, 2H, J=6.3 Hz), 3.75 (t, 2H, J=6.0 Hz), 2.99 (s, 6H), 2.00 (pentet, 2H, J=6.2 Hz). ¹³C NMR ((CD₃)₂CO) δ160.2, 151.6, 131.8, 127.4, 122.6, 120.0, 117.7, 116.3, 113.4, 66.2, 59.1, 58.9, 40.6, 33.4. HRMS calcd for C₁₉H₂₃N₄O₂ (M+H⁺), 339.1822; found, 339.1825.

### 2-(4-(4-(4-(dimethylamino)phenyl)-1H-1,2,3-triazol-1-yl)phenoxy)ethyl 4-methylbenzenesulfonate (16a)

To a stirred solution of 15b (0.162 g, 0.5 mmol) in CH₂Cl₂ (5 mL) cooled with ice bath (0° C.) was added Et₃N (0.35 mL, 2.5 mmol), p-toluenesulfonyl chloride (TsCl, 0.143 g, 0.75 mmol) and catalytic amount 4-dimethylaminopyridine (DMAP, 0.005 g). After addition, the reaction mixture was maintained at 0° C. for 10 min. The ice bath was removed, the reaction was maintained at r.t. for 0.5 h and submitted to standard workup (solvent: CHCl₃/MeOH, 90/10). The crude product was purified by FC (CHCl₃/MeOH, 97/3) to provide pale white solid as the compound (0.228 g, 96%). ¹H NMR (CDCl₃) δ8.01 (s, 1H), 7.86-7.79 (m, 4H), 7.67 (d, 2H, J=9.0 Hz), 7.37 (d, 2H, J=8.5 Hz), 6.96-6.91 (m, 4H), 4.44-4.40 (m, 2H), 4.25-4.21 (m, 2H), 3.05 (s, 6H), 2.47 (s, 3H). ¹³C NMR (CDCl₃) δ158.3, 148.8, 145,3, 132,8, 131.3, 130.0, 128.1, 126.9, 122.2, 116.8, 115.6, 113.2, 68.1, 66.0, 40.8, 21.7.

### 3-(4-(4-(4-(dimethylamino)phenyl)-1H-1,2,3-triazol-1-yl)phenoxy)propyl 4-methylbenzenesulfonate (16b)

Following the procedure in the preparation of 16a, compound 16b was prepared from alcohol 15c (0.169 g, 0.5 mmol) as a pale white solid (0.221 g, 90% yield). ¹H NMR ((CD₃)₂CO) δ8.65 (s, 1H), 7.84-7.77 (m, 6H), 7.40 (d, 2H, J=8.0 Hz), 7.03 (dt, 2H, J₁=9.0 Hz, J₂=2.8 Hz), 6.83 (d, 2H, J=8.9 Hz), 4.29 (t, 2H, J=6.0 Hz), 4.08 (t, 2H, J=5.9 Hz), 2.99 (s, 6H), 2.38 (s, 3H), 2.16 (pentet, 2H, J=6.0 Hz). ¹³C NMR ((CD₃)₂CO) δ159.6, 151.6, 145.9, 131.0, 128.7, 127.4, 126.1, 122.5, 120.0, 117.7, 116.2, 113.4, 68.2, 64.7, 40.6, 21.6.

### 4-(1-(4-(2-fluoroethoxy)phenyl)-1H-1,2,3-triazol-4-yl)-N,N-dimethylbenzenamine (17a)

To a solution of tosylate 16a (0.096 g, 0.20 mmol) in THF (1 mL) was added TBAF solution (1.0 M in THF, 1.0 mL). The reaction solution was heated in microwave reactor at 110° C. for 0.5 h. After cooling and standard workup with EtOAc, the residue was purified by PTLC (EtOAc/Hexanes, 50/50) to afford a light brown solid 8c (0.052 g, 80%). ¹H NMR ((CD₃)₂CO) δ 8.65 (s, 1H), 7.90-7.78 (m, 4H), 7.20 (dt, 2H, J₁=9.1 Hz, J₂=2.8 Hz), 6.83 (d, 2H, J=8.9 Hz), 4.96-4.923 (m, 1H), 4.72-4.68 (m, 1H), 4.48-4.44 (m, 1H), 4.33-4.29 (m, 1H), 3.00 (s, 6H). ¹³C NMR ((CD₃)₂CO) δ159.6, 151.7, 149.4, 132.3, 127.4, 122.6, 120.0, 117.7, 116.4, 113.4, 84.6, 81.3, 69.0, 68.6, 40.6. HRMS calcd for C₁₈H₁₉FN₄O (M⁺), 326.1543; found, 326.1532.

### 4-(1-(4-(3-fluoropropoxy)phenyl)-1H-1,2,3-triazol-4-yl)-N,N-dimethylbenzenamine (17b)

Following the procedure in the preparation of 17a, compound 17b was prepared from tosylate 16b (0.099 g, 0.2 mmol) as a white solid (0.068 g, 100% yield). ¹H NMR ((CD₃)₂CO) δ 8.64 (s, 1H), 7.88-7.78 (m, 6H), 7.18 (dt, 2H, J₁=6.8 Hz, J₂=2.8 Hz), 6.83 (d, 2H, J=8.9 Hz), 4.79 (t, 1H, J=5.9 Hz), 4.56 (t, 1H, J=5.9 Hz), 4.23 (t, 2H, J=6.2 Hz), 2.99 (s, 6H), 2.28 (pentet, 1H, J=6.0 Hz), 2.15 (pentet, 1H, J=6.0 Hz). ¹³C NMR ((CD₃)₂CO) δ 159.8, 151.6, 149.4, 132.1, 127.4, 122.6, 120.0, 117.7, 116.3, 113.4, 83.1, 79.9, 65.0, 40.6, 31.4, 31.0. HRMS calcd for C₁₉H₂₂FN₄O (M+H⁺), 341.1779; found, 341.1776.

### Example 2

### Radioiodination

Radioiodinated compounds, [¹²⁵I]10a and 10b, were prepared via iododestannylation reactions from the corresponding tributyltin precursors 11a and 11b according to the method described previously. Hydrogen peroxide (50 μL, 3% w/v) was added to a mixture of 50 μL of the tributyltin precursor (4 μg/μL EtOH), 50 μL of 1N HCl and [¹²⁵I]NaI (1-5 mCi purchased from Perkin Elmer) in a sealed vial. The reaction was allowed, to proceed for 10 min at room temperature and terminated by addition of 100 μL of sat. NaHSO₃. The reaction mixture neutralized with 1.5 mL of saturated sodium bicarbonate solution was loaded on a small pre-conditioned C-4 mini-column. After sequential rinsing with 10%, 20% ethanol solution, the desired product [¹²⁵I]10a and 10b were obtained. The radiochemical purity was checked by HPLC using a reversed-phase column (Phenomenex Gemini C18 analytical column, 4.6×250 mm, 5 μm, CH₃CN/ammonium formate buffer (1 mM) 8/2; flow rate 0.5 mL/min). The no-carrier-added products were stored at −20° C. up to 6 weeks for animal studies, homogenate binding and autoradiography studies.

### Radiofluorination

[¹⁸F]Fluoride was produced by the JSW typeBC3015 cyclotron using ¹⁸O(p,n)¹⁸F reaction and passed through a Sep-Pak Light QMA cartridge (Waters) is an aqueous solution in [¹⁸O]-enriched water. The cartridge was dried by airflow, and the ¹⁸F activity was eluted with 1.3 mL of Kryptofix 222 (K222)/K₂CO₃ solution (11 mg of K222 and 2.6 mg of K₂CO₃ in CH₃CN/H₂O 1.12/0.18). The solvent was removed at 120° C. under an argon stream. The residue was azeotropically dried with 1 mL of anhydrous CH₃CN twice at 120° C. under a nitrgon stream. A solution of tosylate precursor 16a or 16b (2 mg) in DMSO (0.2 mL) was added to the reaction vessel containing the dried ¹⁸F activities. The mixture was heated at 120° C. for 4 min. Water (5 mL) was added and the mixture was passed through preconditioned OASIS HLB cartridge (3 cc) (Waters). The cartridge was washed with 10 mL water and labeled compound was eluted with 2 mL CH₃CN. Eluted compound was purified by HPLC. [Phenonemex Gemini C18 semi-prep column (10×250 mm, 5 μm), CH₃CN/Water 7/3, flow rate 3 mL/min, t_{R}=11 min]. The radiochemical purity and specific activity were determined by analytical HPLC. [Phenomenex Gemini C18 analytical column (4.6×250 mm, 5 μm), CH₃CN/Ammonium formate buffer (10 mM) 8/2; Flow rate 1 mL/min; t_{R}=4.8 min for [¹⁸F]17a, 5.7 min for [¹⁸F]17b). Specific activity was estimated by comparing UV peak intensity of the purified [¹⁸F] labeled compound with reference non-radioactive compound of known concentration.

### Example 3

### Binding Studies

AD postmortem brain tissues were obtained from University of Washington Alzheimer's Disease Research Center. The neuropathological diagnosis was confirmed by current criteria (NIA-Reagan Institute Consensus Group, 1997). Homogenates were then prepared from dissected gray matters from four pooled AD patients in phosphate buffered saline (PBS, pH 7.4) at the concentration of approximately 100 mg wet tissue/ml (motor-driven glass homogenizer with setting of 6 for 30 sec). The homogenates were aliquoted into 1 mL-portions and stored at −70° C. for up to 2 years without loss of binding signal.

Ligand [¹²⁵I]2 with 2,200 Ci/mmol specific activity and greater than 95% radiochemical purity was prepared using the standard iododestannylation reaction, and purified by a simplified C-4 mini-column as described previously. Binding assays were carried out in 12×75 mm borosilicate glass tubes. For competition studies, the reaction mixture contained 50 □L of pooled AD brain homogenates (20-50 μg), 50 μl of [¹²⁵I]2 (0.04-0.06 nM diluted in PBS) and 50 pi of inhibitors (10⁻⁵-10⁻¹⁰ M diluted serially in PBS containing 0.1% bovine serum albumin) in a final volume of 1 mL. Nonspecific binding was defined in the presence of 600 nM 2 in the same assay tubes. The mixture was incubated at 37° C. for 2 h and the bound and the free radioactivity were separated by vacuum filtration through Whatman GF/B filters using a Brandel M-24R cell harvester followed by 2×3 mL washes of PBS at room temperature. Filters containing the bound ¹²⁵I ligand were counted in a gamma counter (Packard 5000) with 70% counting efficiency. Under the assay conditions, the non-specifically bound fraction was less than 20% of the total radioactivity. The results of inhibition experiments were subjected to nonlinear regression analysis using equilibrium binding data analysis which Kᵢ values were calculated.

Similarly, the specific binding of radioiodinated and radiofluorinated ligands (0.06 nM for [¹²⁵I]probe and 0.5 nM for [¹⁸F]probe to homogenates, prepared from the gray and white matters of AD and control brain tissues, were carried out as described above. Nonspecific binding was determined in the presence of 2 μM of the corresponding nonradioactive probes.

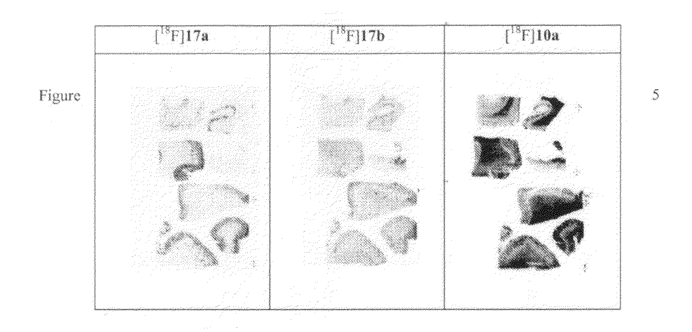

To further characterize the specificity of plaque binding, we evaluated [¹²⁵I]10a and [¹⁸F]17a by a direct in vitro binding assay using homogenates (from different brain regions) prepared from AD and control brain tissues. For both ligands the signal for specific binding was detected predominantly in the gray matter homogenates of AD. In contrast, in white matter homogenates of AD brain, the binding signal for both [¹²⁵I]10a and [¹⁸F]17a were very low or nonexistent (FIG. 5). In the homogenates of control brain tissues (both gray and white matters), the specific binding signal was low suggesting that the specific binding was highly selective to the AD brain only due to the presence of Aβ plaques deposited in these brain samples.

In vitro Autoradiography

To compare different probes using similar sections of human brain tissue, human macro-array brain sections from 6 confirmed AD cases and one control subject were assembled. The presence and localization of plaques on the sections was confirmed with immunohistochemical staining with monoclonal Aβ antibody 4G8 (Sigma). The frozen sections were incubated with [¹²⁵I] and [¹⁸F]tracers (200,000-250,000 cpm/200 μL) for 1 h at room temperature. The sections were then dipped in saturated lithium carbonate in 40% EtOH (two two-minute washes) and washed with 40% EtOH (one two-minute wash) followed by rinsing with water for 30 sec. After drying, the ¹²⁵I- or ¹⁸F-labeled sections were exposed to Kodak Biomax MR film overnight.

### Organ Distribution in Normal Mice

While under isoflurane anesthesia, 0.15 mL of a 0.1% bovine serum albumin solution containing [¹²⁵I] or [¹⁸F]tracers (5-10 μCi) was injected directly into the tail vein of ICR mice (22-25 g, male). The mice (n=3 for each time point) were sacrificed by cervical dislocation at designated time-points post injection. The organs of interest were removed and weighed, and the radioactivity was counted with an automatic gamma counter. The percentage dose per organ was calculated by a comparison of the tissue counts to suitably diluted aliquots of the injected material. The total activity of the blood was calculated under the assumption that it is 7% of the total body weight. The % dose/g of samples was calculated by comparing the sample counts with the count of the diluted initial dose.

### Example 4

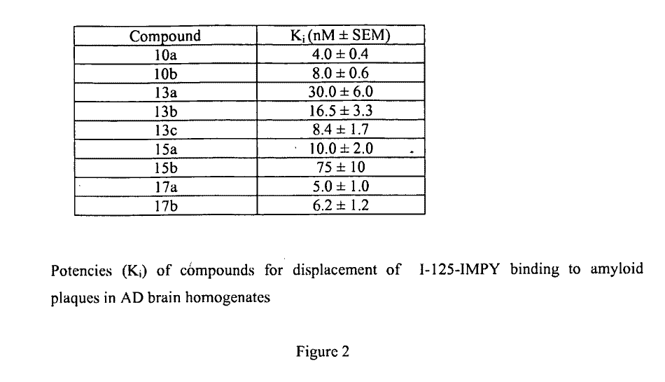

### Film Autoradiography

[¹⁸F]tracers: Brain sections from AD subjects were obtained by freezing the brain in powdered dry ice and cut into 20 micrometer-thick sections. The sections were incubated with [¹⁸F]tracers (200,000-250,000 cpm/200 μl) for 1 hr at room temperature. The sections were then dipped in saturated Li₂CO3 in 40% EtOH (two two-minute washes) and washed with 40% EtOH (one two-minute wash) followed by rinsing with water for 30 sec. After drying, the ¹⁸F-labeled sections were exposed to Kodak MR film overnight. The results are depicted in the film in FIG. 2.

[¹²⁵I]tracers: To compare different probes using similar sections of human brain tissue, human macro-array brain sections from 6 confirmed AD cases and one control subject were assembled. The presence and localization of plaques on the sections was confirmed with immunohistochemical staining with monoclonal Aβ antibody 4G8 (Sigma). The sections were incubated with [¹²⁵I]tracers (200,000-250,000 cpm/200 μL) for 1 h at room temperature. The sections were then dipped in saturated Li₂CO₃ in 40% EtOH (two two-minute washes) and washed with 40% EtOH (one two-minute wash) followed by rinsing with water for 30 sec. After drying, the ¹²⁵I-labeled sections were exposed to Kodak Biomax MR film overnight.

A macro-array block was constructed using postmortem human brain samples consisting of seven confirmed AD cases. After sectioning of this macro-array block, adjacent sections, which reflect a comparable pathophysiology, were used. In vitro film autoradiography was carried out using these novel ¹²⁵I or ¹⁸F labeled diphenyltriazole probes. Among the probes examined, [¹⁸F]17a and [¹⁸F]17b exhibited the most distinctive Aβ plaque-labeling and a minimal level of background in the white matter areas of AD brain (FIG. 5). The labeling pattern was consistent with that observed by immunohistochemical labeling with an antibody (4G8) specific for Aβ (data not shown). In addition to plaque labeling, [¹²⁵I]10a, displayed a significant white matter labeling (FIG. 5) under a similar incubation condition. Similar pattern was also observed for the radioiodinated N,N′-dimethylated probe [¹²⁵I]10b (data not shown). The higher background labeling observed for [¹²⁵I]10a and [¹²⁵I]10b may be related to their higher lipophilicity (log P=3.0 and 3.4, respectively).

### Example 5

### Partition Coefficient

Partition coefficients were measured by mixing the [¹²⁵I] or [¹⁸F]tracer with 3 g each of 1-octanol and buffer (0.1 M phosphate, pH 7.4) in a test tube. The test tube was vortexed for 3 min at room temperature, followed by centrifugation for 5 min. Two weighed samples (0.5 g each) from the 1-octanol and buffer layers were counted in a well counter. The partition coefficient was determined by calculating the ratio of cpm/g of 1-octanol to that of buffer. Samples from the 1-octanol layer were re-partitioned until consistent partitions of coefficient values were obtained. The measurement was done in triplicate and repeated three times.

A moderate lipophilicity (log P=1-3.5) will be considered as a highly desirable property. The radiofluorinated diphenyltriazoles [¹⁸F]17a and [¹⁸F]17b showed lower PC (log P=2.7) as compared to the two radioiodinated triazoles, [¹²⁵I]10a and [¹²⁵I]10b showing a log P of 3.0 and 3.4, respectively.

### Example 6

### Biodistribution

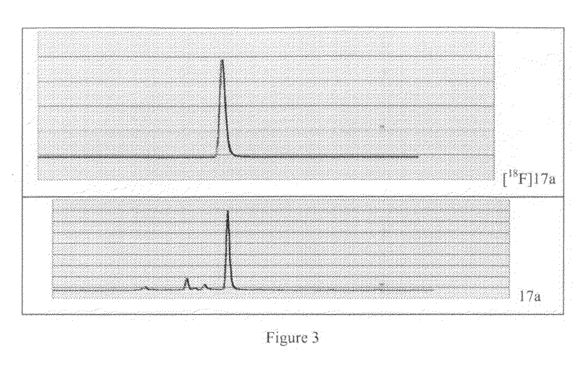

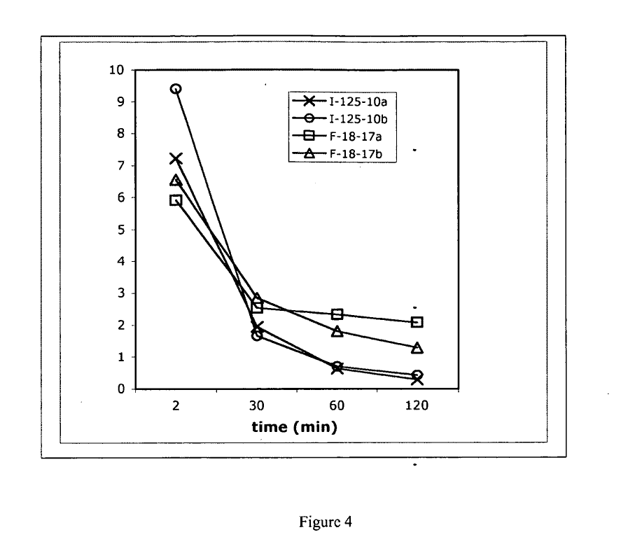

Whole animal distribution evaluated in normal mice for these radiolabeled triazoles displayed excellent initial brain penetrations (6-9.5% dose/g at 2 min) (FIG. 3). The initial high brain uptakes (at 2 min after the injection) observed for these radiolabeled probes, especially for radioiodinated probes [¹²⁵I]10a and [¹²⁵I]10b, were subsequently followed by rapidly washout with less than 0.5% dose/g remaining in the brain at 2 hr after the injection (FIG. 4). [¹⁸F]17a and [¹⁸F]17b appears to show a fast washouts initial, then followed by a gradual and slower rates of clearance from the mouse brain, resulting in a higher residual radioactivity remaining (1-2% dose/g at 2 hr after tracer injection). Thus, the two iodinated probes. [¹²⁵I]10a and [¹²⁵I]10b resulted in higher and better brain washout indexes (brain_{2 min}/brain_{30 min}) of 5.66 and 3.72, respectively which are more favorable compared to [¹⁸F]17a and [¹⁸F]17b with lower indexes of 2.34 and 2.31, respectively for Aβ plaque detection. A significant amount of in vivo defluorination (reflected by bone uptake) observed with [¹⁸F]17a (4.64% dose/g, supporting data) and [¹⁸F]17b (18.59% dose/g, supporting data). These values are 5 to 10 times higher as compared to the values reported for the previous PET ligands. Table 1 below shows the biodistribution data for compound [¹⁸F]17b.

TABLE 1
Biodistribution in ICR mice after iv injection of [¹⁸F]17b in 0.1%
BSA/<1% EtOH in water (% dose/g, avg of 3 mice ± SD)

| Organ | 2 min | 30 min | 1 hr | 2 hr |
|---|---|---|---|---|
| Blood | 3.14 ± 0.46 | 2.50 ± 0.28 | 2.74 ± 0.32 | 1.35 ± 0.10 |
| Heart | 9.47 ± 3.41 | 2.88 ± 0.40 | 2.35 ± 0.29 | 1.04 ± 0.17 |
| Muscle | 1.18 ± 0.34 | 1.49 ± 0.08 | 1.43 ± 0.13 | 0.66 ± 0.07 |
| Lung | 10.66 ± 1.26 | 3.79 ± 0.68 | 2.88 ± 0.21 | 1.23 ± 0.04 |
| Kidney | 15.54 ± 2.94 | 5.02 ± 0.75 | 3.74 ± 0.42 | 1.46 ± 0.17 |
| Spleen | 3.70 ± 1.29 | 2.53 ± 0.37 | 2.13 ± 0.18 | 0.92 ± 0.11 |
| Liver | 15.82 ± 2.23 | 13.10 ± 1.25 | 8.68 ± 0.58 | 3.50 ± 0.82 |
| Skin | 0.88 ± 0.29 | 1.97 ± 0.14 | 1.73 ± 0.18 | 0.75 ± 0.03 |
| Brain | 10.28 ± 1.79 | 1.61 ± 0.16 | 1.68 ± 0.20 | 0.99 ± 0.13 |
| Bone | 1.91 ± 0.63 | 1.69 ± 0.27 | 2.55 ± 0.50 | 2.67 ± 0.07 |

### Example 7

### 2-(4-(4-(4-(dimethylamino)phenyl)-1H-1,2,3-triazol-1-yl)-2-iodophenoxy)ethanol

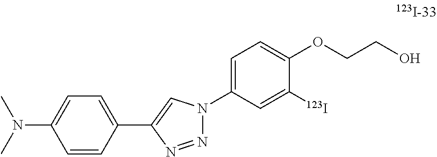

### 2-(4-(4-(4-(dimethylamino)phenyl)-1H-1,2,3-triazol-1-yl)-2-[¹²³I]iodophenoxy)ethanol

### Summary

¹²³I-33 is a novel tracer that may be useful for SPECT imaging of amyloid-β (Aβ) pathology (the chief constituent of amyloid plaques) in patients with cognitive impairment suspected to be due to Alzheimer's disease (AD). Because SPECT agents labeled with ¹²³I have long half-lives (approximately 13 hours) they can be prepared centrally, reducing the potential cost and variability involved with regional or on-site radiosynthesis. These inherent advantages, together with the excellent amyloid binding properties of ¹²³I-33, provide a strong rationale for testing ¹²³I-33 as an Aβ imaging agent.

Compound 33 shows high affinity and specific binding to amyloid plaques, as demonstrated by competitive binding studies using the known amyloid binding agent ¹²⁵I-IMPY (6-iodo-2-(4′-dimethylamino-)phenyl-imidazo[1,2-a]pyridine). In these experiments Compound 33 showed a Ki of 7.5±0.5 nM, comparable to other experimental amyloid imaging agents. ¹²³I-33, when applied at tracer concentrations, specifically labeled Aβ plaques in sections from patients with pathologically confirmed AD.

TABLE 2
Pharmacological properties of Compound 33

| | | Binding | | Biodistribution in | |
|---|---|---|---|---|---|
| | Binding | Affinity | | Mice (% dose/g in brain) | |
| | Target | (Ki, nM) | Selectivity | 2 min | 60 min |
| Compound | Amyloid | 7.5 ± 0.5 | No high | 3.69 ± 0.14 | 0.25 ± 0.03 |
| 33 | Plaque | | affinity | | |
| | | | binding to any | | |
| | | | CNS receptors | | |

In mouse in-vivo experiments ¹²³I-33 showed appropriate biodistribution for a brain imaging agent. When administered i.v. to male mice, ¹²³I-33 entered the brain quickly and reached a peak brain concentration of 3.7% dose/g within 2 min post administration and fell to 0.25% dose/g within 60 min. Similar results were seen in female mice. An estimate of human dosimetry, based on extrapolation from the mouse data, suggests that the dose-limiting organs (assuming thyroid blocking) will be the intestines. At the proposed 5 mCi human dose the lower large intestine is estimated to receive approximately 3.8 rem, while the upper large intestine is estimated to receive approximately 3.3 rem of exposure. The estimated human effective dose (ED) of approximately 1.26 rem is comparable to that for recommended doses of other ¹²³I-labeled SPECT imaging radiopharmaceuticals such as ¹²³I-MIBG, ¹²³I-IMPY, and ¹²³I-βCIT.

### Binding Affinity of Compound 33 in AD Brain Homogenates, as Measured by Inhibition of ¹²⁵I-IMPY Binding

Postmortem brain tissue was obtained and neuropathological diagnosis was confirmed in accordance with the NIA-Reagan Institute Consensus Group criteria. Homogenates were then prepared from dissected gray matter, pooled in phosphate buffered saline and aliquoted into 1-ml portions (100 mg wet tissue/ml), which could be stored at −70° C. for 3-6 months without loss of binding signal.

For the binding assays brain homogenates were incubated with ¹²⁵I-IMPY (0.04-0.06 nM diluted in phosphate buffered saline (PBS)) and test compound (10⁻⁵-10⁻¹⁰ M diluted in PBS containing 0.1% bovine serum albumin (BSA)). Nonspecific binding was defined in the presence of IMPY (600 nM). The bound and free radioactivity was separated by vacuum filtration followed by 2×3 ml washes of PBS. Filters containing the bound ¹²⁵I ligand were assayed in a gamma counter.

Compound 33 potently inhibited ¹²⁵I-IMPY binding in this assay, with a Ki=7.5±0.5 nM. This is comparable to the Ki values published for other presumed amyloid imaging agents that have been tested in humans (Table 3).

TABLE 3
Binding affinity of Compound 33 and other amyloid plaque
ligands to AD brain homogenates (Ki vs. ¹²⁵I-IMPY)

| | Compound | | |
|---|---|---|---|
| | 33 | PIB | FDDNP |
| Ki (nM) ± SD | 7.5 ± 0.5 | 2.8 ± 0.5 | 239 |

PIB = N-methyl[¹¹C]2-4′-methylaminophenyl-6-hydroxybenzathiazole (Pittsburgh Compound B)
FDDNP = 2-(1-{6-[(2-fluoroethyl)(methyl)amino]-2-naphthyl}ethylidene)malononitrile

### Organ Distribution in Normal Mice

While under isoflurane anesthesia, 0.15 ml of a 0.9% saline solution containing ¹²³I-33 (5-10 μCi) was injected directly into the tail vein of ICR mice (22-25 g, male) The mice (n=3 for each time point per sex) were sacrificed, the organs of interest were removed, weighed, and assayed with an automatic gamma counter. The percentage dose per organ was calculated by a comparison of the tissue counts to suitably diluted aliquots of the injected material. Total activities of blood were calculated under the assumption that they were 7% of the total body weight. The % dose/g of samples was calculated by comparing the sample counts with the count of the diluted initial dose. The results (Table 4) suggest ¹²³I-33 readily penetrated and was rapidly cleared from the brain of normal mice.

TABLE 4
Biodistribution of ¹²³I-33 after an i.v. injection in normal male
mice (average of 3 mice ± SD) % dose/organ and % dose/g of blood,
muscle, skin and bone were calculated under the assumption that these
tissues represent 7%, 40%, 15% and 14% of body mass respectively.

| | | | | |
|---|---|---|---|---|
| Organ | 2 min | 60 min | 120 min | 180 min |

% dose/organ

| | | | | |
|---|---|---|---|---|
| Blood | 6.06 ± 0.64 | 5.94 ± 1.05 | 3.95 ± 1.76 | 3.97 ± 1.09 |
| Heart | 0.81 ± 0.13 | 0.15 ± 0.02 | 0.10 ± 0.04 | 0.11 ± 0.04 |
| Muscle | 24.55 ± 1.12 | 6.80 ± 1.30 | 4.02 ± 1.42 | 5.08 ± 1.29 |
| Lung | 1.78 ± 0.30 | 0.48 ± 0.07 | 0.40 ± 0.17 | 0.40 ± 0.04 |
| Kidney | 5.39 ± 0.62 | 1.37 ± 0.35 | 0.72 ± 0.18 | 0.69 ± 0.08 |
| Spleen | 0.41 ± 0.12 | 0.12 ± 0.01 | 0.08 ± 0.03 | 0.11 ± 0.03 |
| Liver | 22.31 ± 3.90 | 5.84 ± 1.65 | 4.09 ± 0.15 | 4.71 ± 1.52 |
| Skin | 3.90 ± 0.64 | 9.08 ± 2.27 | 4.58 ± 1.55 | 6.09 ± 1.75 |
| Brain | 1.81 ± 0.16 | 0.13 ± 0.01 | 0.06 ± 0.02 | 0.09 ± 0.02 |
| Bone | 6.78 ± 1.92 | 4.73 ± 1.53 | 3.31 ± 2.20 | 3.20 ± 1.33 |
| Thyroid | 0.08 ± 0.03 | 2.56 ± 1.20 | 2.36 ± 0.34 | 5.75 ± 0.99 |
| Pancreas | 0.90 ± 0.12 | 0.25 ± 0.11 | 0.19 ± 0.09 | 0.22 ± 0.03 |
| Stomach | 1.28 ± 0.08 | 14.41 ± 5.98 | 9.58 ± 3.80 | 12.19 ± 0.31 |
| Intestine | 9.07 ± 0.36 | 28.98 ± 11.73 | 45.43 ± 7.34 | 37.88 ± 5.79 |
| Urogenital system | 0.89 ± 0.18 | 3.17 ± 1.86 | 2.48 ± 2.34 | 3.41 ± 3.71 |
| Testes | 0.23 ± 0.04 | 0.25 ± 0.02 | 0.14 ± 0.06 | 0.18 ± 0.03 |
| Tail | 9.42 ± 2.80 | 1.52 ± 0.23 | 0.66 ± 0.13 | 0.76 ± 0.15 |
| Fat | 0.63 ± 0.25 | 0.28 ± 0.05 | 0.32 ± 0.13 | 0.22 ± 0.06 |
| Carcass | 31.71 ± 2.57 | 17.81 ± 3.15 | 11.01 ± 2.84 | 12.79 ± 2.92 |
| % total counted | 90.41 ± 5.38 | 81.43 ± 5.66 | 80.05 ± 6.33 | 81.95 ± 4.47 |

% dose/g

| | | | | |
|---|---|---|---|---|
| Blood | 3.05 ± 0.36 | 2.88 ± 0.53 | 1.83 ± 0.85 | 1.82 ± 0.48 |
| Heart | 5.25 ± 0.62 | 1.17 ± 0.15 | 0.72 ± 0.29 | 0.78 ± 0.21 |
| Muscle | 2.16 ± 0.14 | 0.58 ± 0.12 | 0.33 ± 0.12 | 0.41 ± 0.09 |
| Lung | 9.04 ± 1.83 | 2.68 ± 0.32 | 1.97 ± 0.71 | 1.88 ± 0.19 |
| Kidney | 10.47 ± 0.94 | 2.77 ± 0.58 | 1.36 ± 0.35 | 1.31 ± 0.21 |
| Spleen | 4.24 ± 0.83 | 1.36 ± 0.28 | 0.79 ± 0.27 | 1.08 ± 0.35 |
| Liver | 12.11 ± 2.58 | 3.54 ± 1.27 | 2.32 ± 0.29 | 2.55 ± 0.59 |
| Skin | 0.92 ± 0.15 | 2.05 ± 0.53 | 0.99 ± 0.35 | 1.30 ± 0.36 |
| Brain | 3.69 ± 0.14 | 0.25 ± 0.03 | 0.13 ± 0.04 | 0.18 ± 0.04 |
| Bone | 1.71 ± 0.50 | 1.15 ± 0.38 | 0.77 ± 0.52 | 0.73 ± 0.28 |
| Thyroid | 7.56 ± 1.50 | 242.28 ± 136.52 | 187.40 ± 30.16 | 411.21 ± 52.18 |
| Pancrease | 5.32 ± 0.08 | 1.35 ± 0.52 | 1.09 ± 0.46 | 1.29 ± 0.14 |
| Stomach | 2.77 ± 0.54 | 33.35 ± 9.20 | 14.51 ± 7.23 | 26.14 ± 7.11 |
| Intestine | 3.60 ± 0.39 | 10.67 ± 3.91 | 15.49 ± 1.99 | 14.48 ± 2.45 |
| Urogenital system | 2.01 ± 0.73 | 6.49 ± 2.55 | 5.49 ± 4.77 | 6.31 ± 5.26 |
| Testes | 1.14 ± 0.18 | 1.11 ± 0.21 | 0.66 ± 0.24 | 0.84 ± 0.20 |
| Tail | 13.82 ± 5.45 | 1.97 ± 0.20 | 0.92 ± 0.20 | 1.06 ± 0.18 |
| Fat | 1.68 ± 0.75 | 0.82 ± 0.16 | 0.57 ± 0.27 | 0.56 ± 0.22 |
| Carcass | 1.88 ± 0.15 | 1.05 ± 0.18 | 0.61 ± 0.17 | 0.70 ± 0.17 |

% dose/organ of blood, muscle, skin and bone were calculated under the assumption that they were 7%, 40 15% and 14%.
(Blood: 7%, Muscle: 40%, Skin: 15%, Bone: 14%)

### Partition Coefficient

Partition coefficients were measured by mixing ¹²³I-33 with 3 g each of 1-octanol and buffer (0.1 M phosphate, pH 7.4) in a test tube. The test tube was vortexed for 3 min at room temperature, followed by centrifugation for 5 min. Two weighed samples (0.5 g each) from the 1-octanol and the buffer layer were counted in a well counter. The partition coefficient was determined by calculating the ratio of cpm/g of 1-octanol to that of the buffer. Samples from the 1-octanol layer were repartitioned until consistent partitions of,coefficient values were obtained from the third partition. The measurement was done in triplicate and repeated three times. The partition coefficient for ¹²³I-33 was determined to be 1788 (log P=3.25).

### Example 8

### Biodistribution in ICR Mice After an iv Injection of [¹⁸F]17b in 0.1% BSA/<1% Ethanol in Water

| | | | | |
|---|---|---|---|---|
| Organ | 2 min | 30 min | 1 hr | 2 hr |

(% dose/organ, avg of 3 mice ± SD)

| | | | | |
|---|---|---|---|---|
| Blood | 4.62 ± 0.37 | 3.74 ± 0.14 | 4.33 ± 0.49 | 2.04 ± 0.21 |
| Heart | 0.91 ± 0.28 | 0.30 ± 0.04 | 0.24 ± 0.03 | 0.11 ± 0.01 |
| Muscle | 10.01 ± 2.92 | 12.79 ± 0.74 | 12.87 ± 1.11 | 5.73 ± 0.34 |
| Lung | 1.76 ± 0.15 | 0.67 ± 0.08 | 0.53 ± 0.03 | 0.22 ± 0.00 |
| Kidney | 4.71 ± 0.29 | 1.64 ± 0.24 | 1.34 ± 0.23 | 0.51 ± 0.06 |
| Spleen | 0.36 ± 0.14 | 0.26 ± 0.02 | 0.23 ± 0.03 | 0.09 ± 0.01 |
| Liver | 17.24 ± 0.83 | 14.03 ± 0.92 | 9.64 ± 0.61 | 3.89 ± 0.54 |
| Skin | 2.76 ± 0.79 | 6.31 ± 0.23 | 5.87 ± 0.55 | 2.43 ± 0.20 |
| Brain | 4.45 ± 0.62 | 0.74 ± 0.06 | 0.78 ± 0.08 | 0.44 ± 0.05 |
| Bone | 5.69 ± 2.05 | 5.03 ± 0.38 | 8.04 ± 1.53 | 8.12 ± 0.94 |

(% dose/g, avg of 3 mice ± SD)

| | | | | |
|---|---|---|---|---|
| Blood | 3.14 ±0.46 | 2.50 ± 0.28 | 2.74 ± 0.32 | 1.35 ± 0.10 |
| Heart | 9.47 ± 3.41 | 2.88 ± 0.40 | 2.35 ± 0.29 | 1.04 ± 0.17 |
| Muscle | 1.18 ± 0.34 | 1.49 ± 0.08 | 1.43 ± 0.13 | 0.66 ± 0.07 |
| Lung | 10.66 ± 1.26 | 3.79 ± 0.68 | 2.88 ± 0.21 | 1.23 ± 0.04 |
| Kidney | 15.54 ± 2.94 | 5.02 ± 0.75 | 3.74 ± 0.42 | 1.46 ± 0.17 |
| Spleen | 3.70 ± 1.29 | 2.53 ± 0.37 | 2.11 ± 0.18 | 0.92 ± 0.11 |
| Liver | 15.82 ± 2.23 | 13.10 ± 1.25 | 8.68 ± 0.58 | 3.50 ± 0.82 |
| Skin | 0.88 ± 0.29 | 1.97 ± 0.14 | 1.73 ± 0.18 | 0.75 ± 0.03 |
| Brain | 10.28 ± 1.79 | 1.61 ± 0.16 | 1.68 ± 0.20 | 0.99 ± 0.13 |
| Bone | 1.91 ± 0.63 | 1.69 ± 0.27 | 2.55 ± 0.50 | 2.67 ± 0.07 |

### Example 9

A compound of the present invention is tested in an established in-vitro immunoblot assay for its ability to inhibit the formation of Aβ oligomers and fibrils (Yang F, Liim G P, Begum A N et al. Curcumin inhibits formation of amyloid β oligomers and fibrils, binds plaques, and reduces amyloid in-vivo. J. Biol. Chem. 280:5892-5901, 2005). Curcumin, a natural molecule serves as positive control. Compounds of this invention are able to inhibit the aggregation Aβ in a manner similar to Curcumin at concentrations of 1-100 μM.

It will be understood to those of ordinary skill in the art that the same can be performed within a wide and equivalent range of conditions, formulations, and other parameters without affecting the scope of the invention or any embodiment thereof. All patents, patent applications, and publications cited herein are fully incorporated by reference herein in their entirety.

What is claimed:

1. A compound of Formula I, 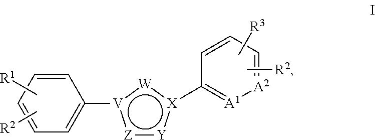
or a pharmaceutically acceptable salt thereof; wherein:
W, Y and Z are each independently CH, N, NH, O or S; provided that at least one of W, Y and Z is N or O;
V and X are independently C or N;
A¹ and A² are independently N, CR³ or CR⁴, as permitted;
R¹ and R² are independently:
—(CH₂)ₚNR^{a}R^{b}, wherein R^{a} and R^{b} are independently hydrogen, (C₁₋₄)alkyl, hydroxy(C₁₋₄)alkyl or halo(C₁₋₄)alkyl, and p is an integer from 0 to 5; hydroxy; (C₁₋₄)alkoxy; hydroxy(C₁₋₄)alkyl; halogen; cyano; hydrogen; nitro; (C₁-C₄)alkyl; halo(C₁-C₄)alkyl; formyl; —NHCO(C₁₋₄ alkyl); —OCO(C₁₋₄ alkyl); or radiohalogen;
R³ is s hydrogen or one of i-vi: 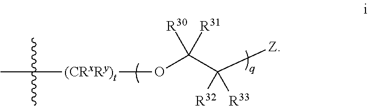
wherein q is an integer from 1 to 10; R^{x} and R^{y} are hydrogen, hydroxy or (C₁₋₄)alkyl; t is 0, 1, 2 or 3; Z is halogen, hydroxy, OTs (tosylate) or amino; and R³⁰, R³¹, R³² and R³³ are in each instance independently hydrogen, hydroxy, (C₁₋₄)alkoxy, (C₁₋₄alkyl, or hydroxy(C₁₋₄alkyl; 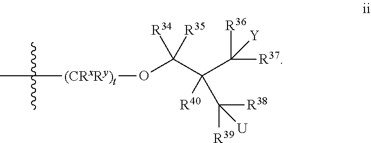
wherein R^{x} and R^{y} are hydrogen, hydroxy or (C₁₋₄)alkyl; t is 0, 1, 2 or 3; Y is halogen, halogen substituted benzoyloxy, halogen substituted phenyl(C₁₋₄alkyl, halogen substituted aryloxy, or halogen substituted (C₆₋₁₀)aryl; U is hydrogen, hydroxy, halogen, halogen substituted benzoyloxy, halogen substituted phenyl(C₁₋₄)alkyl, halogen substituted aryloxy, or halogen substituted C₆₋₁₀ aryl; and R³⁴, R³⁵, R³⁶, R³⁷, R³⁸, R³⁹ and R⁴⁰ are in each instance independently hydrogen, halogen, hydroxy, (C₁₋₄)alkoxy, (C₁₋₄)alkyl, or hydroxy(C₁₋₄)alkyl;
iii. NR′R″, wherein at least one of R′ and R″ is (CH₂)_{d}X, where X is halogen, preferably F or ¹⁸F, and d is an integer from 1 to 4; the other of R′ and R″ is hydrogen, (C₁₋₄)alkyl, halo(C₁₋₄)alkyl, or hydroxy(C₁₋₄)alkyl;
iv. NR′R″—(C₁₋₄)alkyl, wherein at least one of R′ and R″ is (CH₂)_{d}X, where X is halogen, preferably F or ¹⁸F, and d is an integer from 1 to 4; the other of R′ and R″ is hydrogen, (C₁₋₄)alkyl, halo(C₁₋₄)alkyl, or hydroxy(C₁₋₄)alkyl;
v. halo(C₁₋₄)alkyl;
vi. an ether (R—O—R) having the structure: [halo(C₁₋₄)alkyl-O—(C₁₋₄)alkyl]-;
and
R⁴ is hydrogen, halogen, radiohalogen or —(C₁₋₄ alkyl)₃Sn;
provided that one of R³ and R⁴ is other than hydrogen.

2. The compound of claim 1 wherein V, W and X are each N, having the Formula I′:

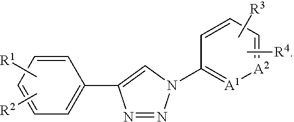

3. The compound of claim 1, wherein R² is hydrogen, halogen or radiohalogen.

4. The compound of claim 1, wherein R² is hydrogen.

5. The compound of claim 1, wherein R¹ is hydroxy or NR^{a}R^{b}(CH₂)ₚ—, wherein R^{a} and R^{b} are independently hydrogen or (C₁₋₄)alkyl and p is 0.

6. The compound of claim 1, wherein R³ is

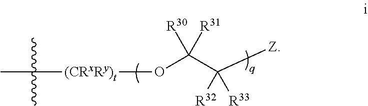

7. The compound of claim 6, wherein q is an integer from 1 to 10.

8. The compound of claim 6, wherein q is 1, 2 or 3.

9. The compound of claim 6, wherein t is 0.

10. The compound of claim 6, wherein R³⁰, R³¹, R³² and R³³ are, in each instance, hydrogen.

11. The compound of claim 1, wherein one of W, Y and Z is O.

12. The compound of claim 1 that is:

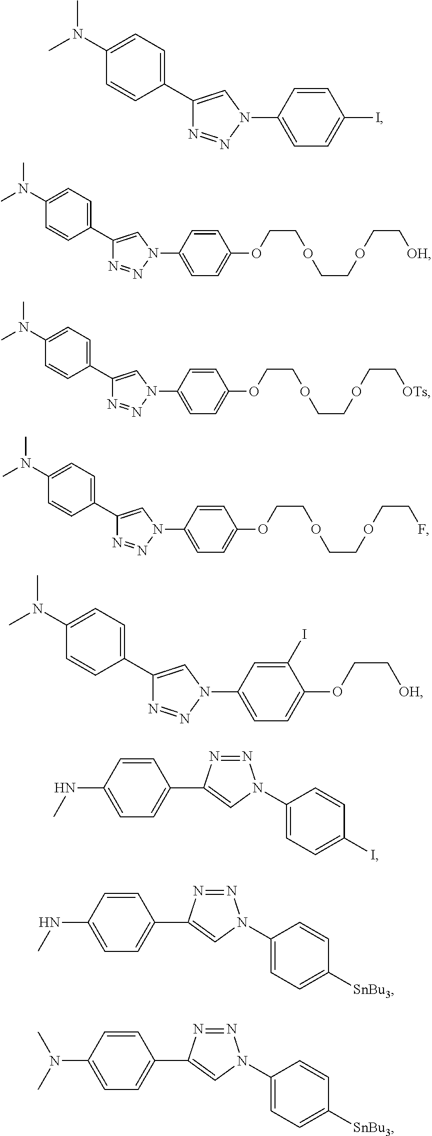

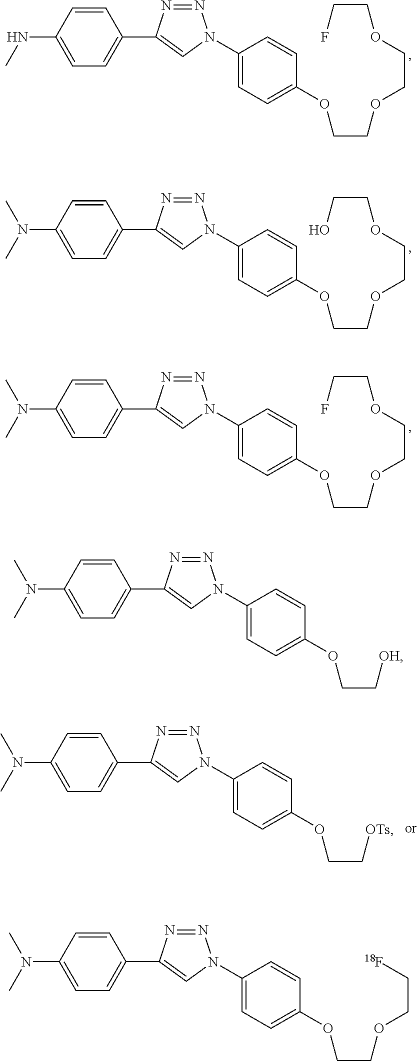

13. The compound of claim 12, wherein the compound comprises a radiohalogen.

14. The compound of claim 1, wherein R³ is

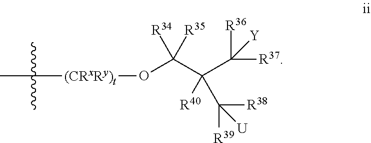

15. The compound of claim 14, wherein R³⁴, R³⁵, R³⁶ and R³⁷, R³⁸, R³⁹ and R⁴⁰ are, in each instance, hydrogen, and t is 0.

16. The compound of claim 15, wherein Y is hydroxy and U is halogen.

17. A compound having the following structure: 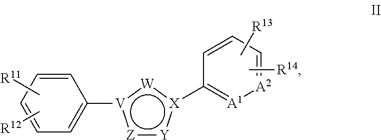
or a pharmaceutically acceptable salt thereof, wherein:
W, Y and Z are each independently CH, N, NH, O or S; provided that at least one of W, Y and Z is N or O;
V and X are independently C or N;
A¹ and A² are independently N, CR¹³ or CR¹⁴ as permitted;
R¹¹ and R¹² are independently:
—(CH₂)ₚNR^{a}R^{b}, wherein R^{a} and R^{b} are independently hydrogen, C₁₋₄ alkyl, hydroxy(C₁₋₄)alkyl or halo(C₁₋₄)alkyl, and p is an integer from 0 to 5; hydroxy; (C₁₋₄)alkoxy; hydroxy(C₁₋₄)alkyl; halogen; cyano; hydrogen; nitro; (C_{1-C}₄)alkyl; halo(C₁-C₄)alkyl; formyl; —NHCO(C₁₋₄ alkyl), or —OCO(C₁₋₄ alkyl);
R¹⁴ is hydrogen;
R¹³ is: 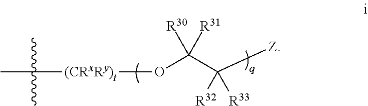
wherein q is an integer from 1 to 10, R^{x} and R^{y} are hydrogen, hydroxy or (C₁₋₄)alkyl; t is 0, 1, 2 or 3; and Z is -Ch; 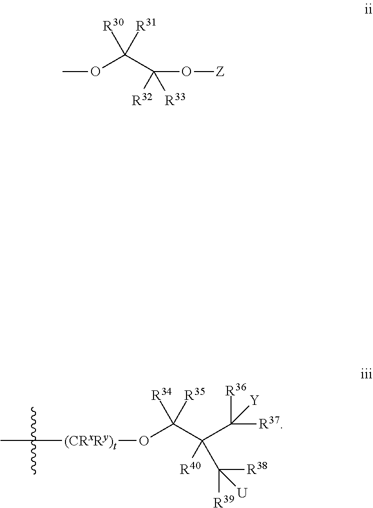
wherein R^{x} and R^{y} are hydrogen, hydroxy or C₁₋₄ alkyl; t is 0, 1, 2 or 3; Y is -Ch; U is selected from the group consisting of hydrogen, hydroxy, halogen, halogen substituted benzoyloxy, halogen substituted phenyl(C₁₋₄)alkyl, halogen substituted aryloxy, or halogen substituted (C₆₋₁₀)aryl; and R³⁴, R³⁵, R³⁶, R³⁷, R³⁸, R³⁹ and R⁴⁰ are in each instance independently hydrogen, halogen, hydroxy, (C₁₋₄)alkoxy, C₁₋₄ alkyl, and hydroxy(C₁₋₄)alkyl;
iv. —(CH₂)_{w}—O-Ch, wherein w is an integer from 1 to 10;
v. -Ch;
vi. —(CH₂)_{w}-Ch, wherein w is an integer from 1 to 10;
wherein, the moiety “-Ch” is a chelating ligand capable of complexing with a metal to form a metal chelate.

18. The compound of claim 17 wherein, said -Ch moiety has the following structure: 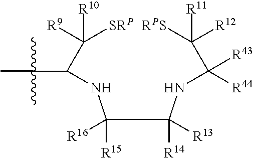
wherein R^{P} is hydrogen or a sulfhydryl protecting group, and R⁹ R¹⁰, R¹¹, R¹², R¹³, R¹⁴, R¹⁵, R¹⁶, R⁴³ and R⁴⁴ are in each instance independently hydrogen, hydroxy, amino, methylamino, dimethylamino, (C₁₋₄)alkoxy, (C₁₋₄)alkyl, or hydroxy(C₁₋₄)alkyl.

19. A radiometal complex of a compound of claim 17, wherein said -Ch has the following structure:

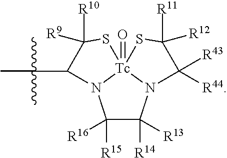

20. The compound of claim 1, having the following structure:

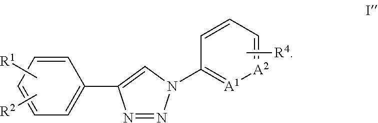

21. The compound of claim 1, having the following general structure:

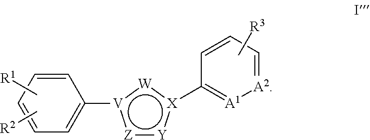

22. A pharmaceutical composition comprising a compound of any one of claims 1-21.

23. A diagnostic composition for imaging amyloid deposits, comprising a radiolabeled compound of any one of claims 1-21.

24. A method of imaging amyloid deposits, comprising:
a. introducing into a mammal a detectable quantity of a diagnostic composition of claim 23;
b. allowing sufficient time for the labeled compound to be associated with amyloid deposits; and
c. detecting the labeled compound associated with one or more amyloid deposits.

25. A method of inhibiting amyloid plaque aggregation in a mammal, comprising administering a composition of claim 22 in an amount effective to inhibit amyloid plaque aggregation
